(19) United States
(12) Patent Application Publication
(10) Pub. No.: US 2007/0066636 A1
(43) Pub. Date: Mar. 22, 2007

(54) POLYMORPHS OF TARTRATE SALT OF 2-[2-(3-(R)-AMINO-PIPERIDIN-1-YL)-5-FLUORO-6-OXO-6H-PYRIMIDIN-1-YLMETHYL]-BENZONITRILE AND METHODS OF USE THEREFOR

(76) Inventors: Leonard J. Chyall, Lafayete, IN (US); Keith Lorimer, West Lafayete, IN (US); Linda J. McClausland, West Lafayette, IN (US)

Correspondence Address: TAKEDA SAN DIEGO, INC., 10410 SCIENCE CENTER DRIVE, SAN DIEGO, CA, 92121, US

(21) Appl. No.: 11/531,638

(22) Filed: Sep. 13, 2006

Related U.S. Application Data
(60) Provisional application No. 60/718,164, filed on Sep. 16, 2005.

(51) Int. Cl. A61K 31/513; C07D 403/04

(52) U.S. Cl. 514/269; 544/310

(57) ABSTRACT

Compositions comprising Compound I, wherein the Compound I is present in one or more polymorphic forms. Also provided are kits and articles of manufacture with compositions comprising one or more polymorphs of Compound I, and methods of using the compositions to treat various diseases.

### RELATED APPLICATION

This application claims the benefit of U.S. Provisional Application No. 60/718,164 filed Sep. 16, 2005, which is incorporated herein by reference.

### FIELD OF THE INVENTION

The present invention relates generally to polymorphs of the tartrate salt of 2-[2-(3-(R)-amino-piperidin-1-yl)-5-fluoro-6-oxo-6H-pyrimidin-1-ylmethyl]-benzonitrile (referred to herein as “Compound I”); compositions, kits and articles of manufacture comprising polymorphs of Compound I; and methods of their use.

### DESCRIPTION OF RELATED ART

Dipeptidyl Peptidase IV (IUBMB Enzyme Nomenclature EC.3.4.14.5) is a type II membrane protein that has been referred to in the literature by a wide a variety of names including DPP4, DP4, DAP-IV, FAPβ adenosine deaminase complexing protein 2, adenosine deaminase binding protein (ADAbp), dipeptidyl aminopeptidase IV; Xaa-Pro-dipeptidyl-aminopeptidase; Gly-Pro naphthylamidase; postproline dipeptidyl aminopeptidase IV; lymphocyte antigen CD26; glycoprotein GP110; dipeptidyl peptidase IV; glycylproline aminopeptidase; glycylproline aminopeptidase; X-prolyl dipeptidyl aminopeptidase; pep X; leukocyte antigen CD26; glycylprolyl dipeptidylaminopeptidase; dipeptidyl-peptide hydrolase; glycylprolyl aminopeptidase; dipeptidyl-aminopeptidase IV; DPP IV/CD26; amino acyl-prolyl dipeptidyl aminopeptidase; T cell triggering molecule Tp103; X-PDAP. Dipeptidyl Peptidase IV is referred to herein as “DPP-IV”.

DPP-IV is a non-classical serine aminodipeptidase that removes Xaa-Pro dipeptides from the amino terminus (N-terminus) of polypeptides and proteins. DPP-IV dependent slow release of dipeptides of the type X-Gly or X-Ser has also been reported for some naturally occurring peptides.

DPP-IV is constitutively expressed on epithelial and endothelial cells of a variety of different tissues (intestine, liver, lung, kidney and placenta), and is also found in body fluids. DPP-IV is also expressed on circulating T-lymphocytes and has been shown to be synonymous with the cell-surface antigen, CD-26. DPP-IV has been implicated in a number of disease states, some of which are discussed below.

DPP-IV is responsible for the metabolic cleavage of certain endogenous peptides (GLP-1 (7-36), glucagon) in vivo and has demonstrated proteolytic activity against a variety of other peptides (GHRH, NPY, GLP-2, VIP) in vitro.

GLP-1 (7-36) is a 29 amino-acid peptide derived by post-translational processing of proglucagon in the small intestine. GLP-1 (7-36) has multiple actions in vivo including the stimulation of insulin secretion, inhibition of glucagon secretion, the promotion of satiety, and the slowing of gastric emptying. Based on its physiological profile, the actions of GLP-1 (7-36) are believed to be beneficial in the prevention and treatment of type II diabetes and potentially obesity. For example, exogenous administration of GLP-1 (7-36) (continuous infusion) in diabetic patients has been found to be efficacious in this patient population. Unfortunately, GLP-1 (7-36) is degraded rapidly in vivo and has been shown to have a short half-life in vivo (t_{1/2}=1.5 minutes).

Based on a study of genetically bred DPP-IV knock out mice and on in vivo/in vitro studies with selective DPP-IV inhibitors, DPP-IV has been shown to be the primary degrading enzyme of GLP-1 (7-36) in vivo. GLP-1 (7-36) is degraded by DPP-IV efficiently to GLP-1 (9-36), which has been speculated to act as a physiological antagonist to GLP-1 (7-36). Inhibiting DPP-IV in vivo is therefore believed to be useful for potentiating endogenous levels of GLP-1 (7-36) and attenuating the formation of its antagonist GLP-1 (9-36). Thus, DPP-IV inhibitors are believed to be useful agents for the prevention, delay of progression, and/or treatment of conditions mediated by DPP-IV, in particular diabetes and more particularly, type 2 diabetes mellitus, diabetic dislipidemia, conditions of impaired glucose tolerance (IGT), conditions of impaired fasting plasma glucose (IFG), metabolic acidosis, ketosis, appetite regulation and obesity.

DPP-IV expression is increased in T-cells upon mitogenic or antigenic stimulation (Mattem, T., et al., Scand. J. Immunol., 1991, 33, 737). It has been reported that inhibitors of DPP-IV and antibodies to DPP-IV suppress the proliferation of mitogen-stimulated and antigen-stimulated T-cells in a dose-dependant manner (Schon, E., et al., Biol. Chem., 1991, 372, 305). Various other functions of T-lymphocytes such as cytokine production, IL-2 mediated cell proliferation and B-cell helper activity have been shown to be dependent on DPP-IV activity (Schon, E., et al., Scand. J. Immunol., 1989, 29, 127). DPP-IV inhibitors, based on boroProline, (Flentke, G. R., et al., Proc. Nat. Acad. Sci. USA, 1991, 88, 1556) although unstable, were effective at inhibiting antigen-induced lymphocyte proliferation and IL-2 production in murine CD4+ T-helper cells. Such boronic acid inhibitors have been shown to have an effect in vivo in mice causing suppression of antibody production induced by immune challenge (Kubota, T. et al., Clin. Exp. Immun., 1992, 89, 192). The role of DPP-IV in regulating T lymphocyte activation may also be attributed, in part, to its cell-surface association with the transmembrane phosphatase, CD45. DPP-IV inhibitors or non-active site ligands may possibly disrupt the CD45-DPP-IV association. CD45 is known to be an integral component of the T-cell signaling apparatus. It has been reported that DPP-IV is essential for the penetration and infectivity of HIV-1 and HIV-2 viruses in CD4+ T-cells (Wakselman, M., Nguyen, C., Mazaleyrat, J.-P., Callebaut, C., Krust, B., Hovanessian, A. G., Inhibition of HIV-1 infection of CD 26+ but not CD 26-cells by a potent cyclopeptidic inhibitor of the DPP-IV activity of CD 26. Abstract P.44 of the 24^{th} European Peptide Symposium 1996). Additionally, DPP-IV has been shown to associate with the enzyme adenosine deaminase (ADA) on the surface of T-cells (Kameoka, J., et al., Science, 193, 26 466). ADA deficiency causes severe combined immunodeficiency disease (SCID) in humans. This ADA-CD26 interaction may provide clues to the pathophysiology of SCID. It follows that inhibitors of DPP-IV may be useful immunosuppressants (or cytokine release suppressant drugs) for the treatment of among other things: organ transplant rejection; autoimmune diseases such as inflammatory bowel disease, multiple sclerosis and rheumatoid arthritis; and the treatment of AIDS.

It has been shown that lung endothelial cell DPP-IV is an adhesion molecule for lung-metastatic rat breast and prostate carcinoma cells (Johnson, R. C., et al., J. Cell. Biol, 1993, 121, 1423). DPP-IV is known to bind to fibronectin and some metastatic tumor cells are known to carry large amounts of fibronectin on their surface. Potent DPP-IV inhibitors may be useful as drugs to prevent metastases of, for example, breast and prostrate tumors to the lungs.

High levels of DPP-IV expression have also been found in human skin fibroblast cells from patients with psoriasis, rheumatoid arthritis (RA) and lichen planus (Raynaud, F., et al., J. Cell. Physiol., 1992, 151, 378). Therefore, DPP-IV inhibitors may be useful as agents to treat dermatological diseases such as psoriasis and lichen planus.

High DPP-IV activity has been found in tissue homogenates from patients with benign prostate hypertrophy and in prostatosomes. These are prostate derived organelles important for the enhancement of sperm forward motility (Vanhoof, G., et al., Eur. J. Clin. Chem. Clin. Biochem., 1992, 30, 333). DPP-IV inhibitors may also act to suppress sperm motility and therefore act as a male contraceptive agent. Conversely, DPP-IV inhibitors have been implicated as novel for treatment of infertility, and particularly human female infertility due to Polycystic ovary syndrome (PCOS, Stein-Leventhal syndrome) which is a condition characterized by thickening of the ovarian capsule and formation of multiple follicular cysts. It results in infertility and amenorrhea.

DPP-IV is thought to play a role in the cleavage of various cytokines (stimulating hematopoietic cells), growth factors and neuropeptides.

Stimulated hematopoietic cells are useful for the treatment of disorders that are characterized by a reduced number of hematopoietic cells or their precursors in vivo. Such conditions occur frequently in patients who are immunosuppressed, for example, as a consequence of chemotherapy and/or radiation therapy for cancer. It was discovered that inhibitors of dipeptidyl peptidase type IV are useful for stimulating the growth and differentiation of hematopoietic cells in the absence of exogenously added cytokines or other growth factors or stromal cells. This discovery contradicts the dogma in the field of hematopoietic cell stimulation, which provides that the addition of cytokines or cells that produce cytokines (stromal cells) is an essential element for maintaining and stimulating the growth and differentiation of hematopoietic cells in culture. (See, e.g., PCT Intl. Application No. PCT /US93/017173 published as WO 94/03055).

DPP-IV in human plasma has been shown to cleave N-terminal Tyr-Ala from growth hormone-releasing factor and cause inactivation of this hormone. Therefore, inhibitors of DPP-IV may be useful in the treatment of short stature due to growth hormone deficiency (Dwarfism) and for promoting GH-dependent tissue growth or re-growth.

DPP-IV can also cleave neuropeptides and has been shown to modulate the activity of neuroactive peptides substance P, neuropeptide Y and CLIP (Mentlein, R., Dahms, P., Grandt, D., Kruger, R., Proteolytic processing of neuropeptide Y and peptide YY by dipeptidyl peptidase IV, Regul. Pept., 49, 133, 1993; Wetzel, W., Wagner, T., Vogel, D., Demuth, H.-U., Balschun, D., Effects of the CLIP fragment ACTH 20-24 on the duration of REM sleep episodes, Neuropeptides, 31, 41, 1997). Thus DPP-IV inhibitors may also be useful agents for the regulation or normalization of neurological disorders.

A need still exists for DPP-IV inhibitors that have advantageous potency, stability, selectivity, toxicity and/or pharmacodynamics properties and which thus may be used effectively in pharmaceutical compositions to treat disease states by the inhibition of DPP-IV.

### SUMMARY OF THE INVENTION

The tartrate salt of 2-[2-(3-(R)-amino-piperidin-1-yl)-5-fluoro-6-oxo-6H-pyrimidin-1-ylmethyl]-benzonitrile (referred to herein as Compound I) which has the formula: 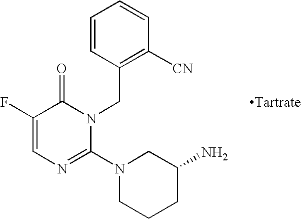
is a DPP-IV inhibitor that is described in U.S. Patent Publication No. US 2005/0070535 (published Mar. 31, 2005 and based on application Ser. No. 10/918,317, filed Aug. 12, 2004). U.S. Patent Publication No. US 2005/0070535 is incorporated herein by reference in its entirety.

The present invention provides novel polymorphs of Compound I, as well as compositions comprising one or more of the novel polymorphs. For ease of reference, the different polymorphs described herein are referred to consistently as Form A through Form F, and amorphous Form 1 and Form 2.

1. Form A

In one embodiment, the present invention relates to a polymorph of Compound I, referred to herein as Form A. Based on its physical properties, Form A is a crystalline form.

Form A may be characterized as having one or more of the following physical characteristics (it being noted that a composition need not necessarily exhibit all of these characteristics in order to indicate the presence of Form A):
(a) may be formed by crystallization from any of the following solvent systems (i) acetone and water, (ii) methanol; (iii) methanol and acetone, (iv) methanol and toluene, and (v) water;

(b) has an X-ray powder diffraction pattern with salient features being major diffraction lines as shown below:

| | °2θ | | | | | | | | | | | | |
|---|---|---|---|---|---|---|---|---|---|---|---|---|---|
| | 6.22 | 7.92 | 8.51 | 10.08 | 12.30 | 12.77 | 13.33 | 13.68 | 14.72 | 15.48 | 16.14 | 17.01 | 18.23 |
| I/I₁ | 39 | 58 | 45 | 60 | 81 | 78 | 33 | 100 | 61 | 18 | 31 | 85 | 50 |

| | °2θ | | | | | | | | | | | |
|---|---|---|---|---|---|---|---|---|---|---|---|---|
| | 18.43 | 18.63 | 19.12 | 20.15 | 20.35 | 21.95 | 22.28 | 22.80 | 23.22 | 23.95 | 24.61 | 25.05 |
| I/I₁ | 27 | 19 | 24 | 36 | 61 | 13 | 25 | 12 | 16 | 24 | 37 | 63 |
| | °2θ | | | | | | | | | | | |
| | 25.39 | 25.67 | 26.33 | 27.05 | 27.65 | 27.94 | 29.23 | 31.05 | 31.31 | 31.76 | 32.44 | 37.14 |
| I/I₁ | 50 | 13 | 20 | 22 | 17 | 24 | 49 | 22 | 21 | 13 | 26 | 24 |

and, in particular, having the following distinguishing peaks:

| | | | | | | | | |
|---|---|---|---|---|---|---|---|---|
| °2θ | 6.22 | 7.92 | 8.51 | 12.77 | 14.72 | 15.48 | 29.23 | 32.44 |

(c) has an IR spectrum comprising absorption peaks at 3478, 3383, 3053, 2946, 2928, 2866, 2843, 2576, 2362, 2338, 2223, 1696, 1671, 1616, 1536, 1487, 1473, 1433, 1407, 1356, 1331, 1321, 1292, 1276, 1263, 1234, 1205, 1156, 1120, 1105, 1074, 1021, 997, 971, 935, 886, 862, 840, 821, 769, 756, 749, 719, 702 and 686 cm⁻¹;
with an IR spectrum comprising unique FT-IR peak positions (peaks that show no other peak within ±4 cm⁻¹ to make up a unique set) at 3478, 3383, 3053, 2928, 2843, 1671, 1105 and 997 cm⁻¹;
(d) has FT-Raman peak positions at 3099, 3077, 3055, 3016, 2994, 2968, 2926, 2914, 2865, 2845, 2222, 1686, 1674, 1604, 1579, 1544, 1487, 1458, 1442, 1404, 1358, 1333, 1277, 1257, 1213, 1185, 1168, 1157, 1077, 1048, 906, 889, 818, 782, 754, 719, 702, 651, 615, 575, 563, 523, 476, 457 and 439 cm⁻¹;
with unique FT-Raman peak positions (peaks that show no other peak within ±4 cm⁻¹ to make up a unique set) at 3099, 3077, 1458, 1333, 889, 702, 651 and 615 cm⁻¹; and/or
(e) has a differential scanning calorimetry spectrum having an endotherm range of about 160° C. to about 185° C., and optionally an endotherm at 172.5° C.
   2. Form B

Form B may be characterized as having one or more of the following physical characteristics (it being noted that a composition need not necessarily exhibit all of these characteristics in order to indicate the presence of Form B):
(a) may be formed by crystallization from any of the following solvent systems (i) tetrahydrofuran, (ii) dioxane and water; and (iii) acetonitrile and water;

(b) has an X-ray powder diffraction pattern with salient features being major diffraction lines as shown below:

| | °2θ | | | | | | | | | | | |
|---|---|---|---|---|---|---|---|---|---|---|---|---|
| | 3.11 | 9.39 | 10.12 | 10.96 | 11.51 | 12.49 | 14.07 | 14.99 | 15.17 | 16.17 | 17.01 | 17.43 |
| I/I₁ | 40 | 10 | 63 | 23 | 100 | 97 | 29 | 13 | 14 | 19 | 20 | 38 |
| | °2θ | | | | | | | | | | | |
| | 18.09 | 18.39 | 18.67 | 19.05 | 20.04 | 20.82 | 21.11 | 21.83 | 22.19 | 22.39 | 22.63 | 23.21 |
| I/I₁ | 23 | 30 | 61 | 31 | 66 | 36 | 17 | 18 | 19 | 24 | 17 | 17 |

| | °2θ | | | | | | | | | | |
|---|---|---|---|---|---|---|---|---|---|---|---|
| | 23.63 | 23.85 | 24.13 | 25.06 | 25.75 | 26.67 | 26.97 | 27.15 | 27.52 | 28.31 | 32.83 |
| I/I₁ | 24 | 35 | 42 | 14 | 15 | 13 | 13 | 20 | 15 | 31 | 16 |

and, in particular, having the following distinguishing peaks:

| | | | | | |
|---|---|---|---|---|---|
| °2θ | 3.11 | 10.96 | 14.07 | 20.04 | 20.82 |

(c) has an IR spectrum comprising absorption peaks at 3376, 3067, 2951, 2866, 2225, 1688, 1613, 1535, 1506, 1478, 1436, 1420, 1376, 1357, 1296, 1262, 1234, 1217, 1194, 1161, 1115, 1071, 1033, 992, 980, 935, 926, 901, 886, 870, 832, 783, 772, 755, 727 and 691 cm⁻¹; and/or
with an IR spectrum comprising unique FT-IR peak positions (peaks that show no other peak within ±4 cm⁻¹ to make up a unique set) at 2951, 1506, 1420, 1217, 1161, 1115 and 1033 cm⁻¹;
(d) has FT-Raman peak positions at 3084, 3056, 2993, 2970, 2926, 2866, 2223, 1686, 1603, 1579, 1540, 1442, 1405, 1357, 1277, 1214, 1184, 1168, 1123, 1071, 1048, 906, 782, 755, 563, 476 and 457 cm⁻¹;
with unique FT-Raman peak positions (peaks that show no other peak within ±4 cm⁻¹ to make up a unique set) at 3084, 2993, 2926, 1686 and 1540 cm⁻¹; and/or
(e) has a differential scanning calorimetry spectrum having an endotherm range of about 100° C. to about 135° C., optionally a range of about 115° C. to about 125° C., and optionally an endotherm at 124.4° C.
   3. Form C

Form C may be characterized as having one or more of the following physical characteristics (it being noted that a composition need not necessarily exhibit all of these characteristics in order to indicate the presence of Form C):
(a) may be formed by crystallization from any of the following solvent systems (i) ethanol and water, and (ii) isopropanol and water;

(b) has an X-ray powder diffraction pattern with salient features being major diffraction lines as shown below:

| | °2θ | | | | | | | | | | | |
|---|---|---|---|---|---|---|---|---|---|---|---|---|
| | 3.10 | 5.22 | 10.17 | 10.47 | 11.42 | 12.49 | 12.71 | 13.50 | 14.29 | 15.39 | 15.68 | 16.26 |
| I/I₁ | 7 | 19 | 49 | 59 | 22 | 26 | 14 | 33 | 42 | 14 | 28 | 16 |
| | °2θ | | | | | | | | | | | |
| | 18.04 | 18.57 | 19.86 | 20.31 | 20.78 | 21.19 | 22.36 | 24.00 | 24.37 | 25.24 | 25.74 | 26.63 |
| I/I₁ | 73 | 35 | 100 | 32 | 20 | 14 | 29 | 54 | 11 | 11 | 11 | 13 |

| | °2θ | | | | | | | | | |
|---|---|---|---|---|---|---|---|---|---|---|
| | 27.05 | 27.82 | 28.22 | 28.93 | 29.58 | 33.51 | 34.03 | 36.63 | 37.69 | 38.14 |
| I/I₁ | 13 | 25 | 10 | 17 | 30 | 14 | 13 | 12 | 15 | 11 |

and, in particular, having the following distinguishing peaks:

| | | | | | |
|---|---|---|---|---|---|
| °2θ | 5.22 | 14.29 | 15.68 | 18.04 | 29.58 |

(c) has an IR spectrum comprising absorption peaks at 3513, 3416, 3064, 2958, 2865, 2668, 2363, 2336, 2239, 2227, 1723, 1692, 1635, 1612, 1587, 1537, 1478, 1432, 1377, 1355, 1297, 1261, 1235, 1212, 1190, 1123, 1071, 1022, 990, 980, 926, 901, 887, 869, 833, 813, 777, 770, 756, 729 and 690 cm⁻¹;
with an IR spectrum comprising unique FT-IR peak positions (peaks that show no other peak within ±4 cm⁻¹ to make up a unique set) at 3513, 2958, 1723, 1635, 1587, 813 and 777 cm⁻¹;
(d) has FT-Raman peak positions at 3082, 3056, 3009, 2972, 2958, 2870, 2844, 2241, 2227, 1944, 1678, 1602, 1581, 1532, 1480, 1436, 1419, 1372, 1357, 1297, 1267, 1217, 1193, 1167, 1071, 1045, 979, 788, 755, 721, 690, 607, 487 and 441 cm⁻¹;
with unique FT-Raman peak positions (peaks that show no other peak within ±4 cm⁻¹ to make up a unique set) at 1944, 1436, 1419, 1372, 1297, 979 and 487 cm⁻¹; and/or
(e) has a differential scanning calorimetry spectrum having an endotherm range of about 100° C. to about 135° C., optionally a range of about 115° C. to about 128° C., and optionally an endotherm at 122.4° C.
   4. Form D

Form D may be characterized as having one or more of the following physical characteristics (it being noted that a composition need not necessarily exhibit all of these characteristics in order to indicate the presence of Form D):
(a) may be formed by crystallization from any of the following solvent systems (i) water, and (ii) methanol;

(b) has an X-ray powder diffraction pattern with salient features being major diffraction lines as shown below:

| | °2θ | | | | | | | | | | | |
|---|---|---|---|---|---|---|---|---|---|---|---|---|
| | 6.21 | 7.90 | 8.50 | 10.07 | 10.58 | 11.34 | 11.83 | 12.31 | 12.75 | 13.31 | 13.66 | 14.71 |
| I/I₁ | 47 | 64 | 47 | 55 | 29 | 18 | 28 | 92 | 80 | 36 | 100 | 38 |
| | °2θ | | | | | | | | | | | |
| | 15.05 | 16.07 | 16.47 | 17.00 | 18.40 | 19.16 | 20.38 | 21.40 | 22.22 | 23.27 | 23.96 | 24.75 |
| I/I₁ | 11 | 32 | 17 | 79 | 64 | 18 | 87 | 34 | 36 | 35 | 31 | 64 |

| | °2θ | | | | | | | | | |
|---|---|---|---|---|---|---|---|---|---|---|
| | 25.13 | 25.45 | 26.40 | 27.02 | 27.77 | 29.22 | 31.15 | 32.42 | 33.13 | 37.16 |
| I/I₁ | 80 | 92 | 27 | 33 | 35 | 42 | 36 | 25 | 27 | 28 |

and, in particular, having the following distinguishing peaks:

| | | | | |
|---|---|---|---|---|
| °2θ | 6.21 | 7.90 | 8.50 | 10.58 |

5. Form E

Form E may be characterized as having one or more of the following physical characteristics (it being noted that a composition need not necessarily exhibit all of these characteristics in order to indicate the presence of Form E):
(a) may be formed by crystallization from any of the following solvent systems (i) water and acetonitrile, and (ii) water and dioxane;

(b) has an X-ray powder diffraction pattern with salient features being major diffraction lines as shown below:

| | °2θ | | | | | | | | | | | | |
|---|---|---|---|---|---|---|---|---|---|---|---|---|---|
| | 5.51 | 9.66 | 11.03 | 11.29 | 12.47 | 13.83 | 14.41 | 16.50 | 16.93 | 18.83 | 19.31 | 20.22 | 20.73 |
| I/I₁ | 36 | 53 | 65 | 18 | 27 | 48 | 47 | 42 | 39 | 59 | 32 | 100 | 33 |
| | °2θ | | | | | | | | | | | | |
| | 21.77 | 22.51 | 24.16 | 25.18 | 25.79 | 26.40 | 26.86 | 27.28 | 27.81 | 28.15 | 29.47 | 33.03 | 34.03 |
| I/I₁ | 10 | 27 | 31 | 16 | 11 | 22 | 16 | 16 | 14 | 39 | 41 | 14 | 16 |

and, in particular, having the following distinguishing peaks:

| | | | | | | | | | | |
|---|---|---|---|---|---|---|---|---|---|---|
| °2θ | 5.51 | 9.66 | 11.03 | 13.83 | 14.41 | 16.50 | 18.83 | 22.51 | 28.15 | 29.47 |

(c) has an IR spectrum comprising absorption peaks at 3440, 3330, 3108, 3067, 2966, 2946, 2864, 2231, 1694, 1614, 1580, 1539, 1491, 1477, 1429, 1410, 1381, 1357, 1295, 1258, 1234, 1189, 1122, 1070, 1023, 991, 980, 923, 889, 868, 837, 770, 756, 728, 690 cm⁻¹;
with an IR spectrum comprising unique FT-IR peak positions (peaks that show no other peak within ±4 cm⁻¹ to make up a unique set) at 3440, 3330, 3108, 1580 and 1381 cm⁻¹;
(d) has FT-Raman peak positions at 3069, 2953, 2871, 2226, 1690, 1601, 1577, 1538, 1481, 1442, 1351, 1286, 1265, 1236, 1212, 1189, 1167, 1071, 1045, 993, 903, 814, 785, 752, 725, 690 and 482 cm⁻¹;
with unique FT-Raman peak positions (peaks that show no other peak within ±4 cm⁻¹ to make up a unique set) at 3069, 1286 and 1236 cm⁻¹; and/or
(e) has a differential scanning calorimetry spectrum having an endotherm range of about 100° C. to about 133° C., optionally a range of about 112° C. to about 126° C., and optionally an endotherm at 121.85° C.
   6. Form F

Form F may be characterized as having one or more of the following physical characteristics (it being noted that a composition need not necessarily exhibit all of these characteristics in order to indicate the presence of Form F):
(a) may be formed by crystallization from water; and/or

(b) has an X-ray powder diffraction pattern with salient features being major diffraction lines as shown below:

| | °2θ | | | | | | | | | | | |
|---|---|---|---|---|---|---|---|---|---|---|---|---|
| | 5.40 | 10.21 | 10.79 | 11.59 | 12.05 | 12.51 | 13.02 | 13.80 | 14.73 | 15.03 | 16.12 | 16.53 |
| I/I₁ | 16 | 67 | 88 | 87 | 16 | 51 | 25 | 10 | 29 | 57 | 75 | 30 |
| | °2θ | | | | | | | | | | | |
| | 17.55 | 17.86 | 18.19 | 18.94 | 19.27 | 19.71 | 20.37 | 20.93 | 21.43 | 22.31 | 22.79 | 23.21 |
| I/I₁ | 36 | 75 | 36 | 47 | 26 | 47 | 86 | 55 | 83 | 30 | 100 | 41 |
| | °2θ | | | | | | | | | | | |
| | 24.13 | 25.07 | 25.46 | 26.15 | 26.70 | 27.27 | 27.71 | 28.07 | 28.73 | 29.92 | 30.81 | 31.82 |
| I/I₁ | 35 | 36 | 57 | 52 | 66 | 22 | 34 | 35 | 48 | 27 | 28 | 24 |

| | °2θ | | | | |
|---|---|---|---|---|---|
| | 32.75 | 33.24 | 33.87 | 36.28 | 36.74 |
| I/I₁ | 17 | 21 | 29 | 19 | 21 |

and, in particular, having the following distinguishing peaks:

| | | | | |
|---|---|---|---|---|
| °2θ | 10.79 | 11.59 | 12.51 | 13.02 |

7. Amporphous Forms 1 and 2

The amorphous forms (Form 1 and Form 2) may be characterized as having one or more of the following physical characteristics (it being noted that a composition need not necessarily exhibit all of these characteristics in order to indicate the presence of the amorphous forms):
(a) may be formed by (i) freeze drying from water (Form 1) and (ii) milling of the salt (Form 2);
(b) has an IR spectrum comprising absorption peaks at 3407, 3064, 2947, 2864, 2226, 1687, 1615, 1536, 1488, 1435, 1351, 1287, 1261, 1235, 1209, 1123, 1074, 1022, 992, 935, 886, 866, 839 and 767 cm⁻¹;
with an IR spectrum comprising unique FT-IR peak positions (peaks that show no other peak within ±4 cm⁻¹ to make up a unique set) at 3407, 1536 and 1287 cm⁻¹;
(c) has FT-Raman peak positions at 3074, 2953, 2870, 2223, 1789, 1691, 1599, 1576, 1536, 1485, 1442, 1398, 1350, 1263, 1210, 1168, 1122, 1100, 1045, 989, 901, 864, 817, 781, 749, 724, 688, 636, 610, 559, 524, 480 and 441 cm⁻¹;
with unique FT-Raman peak positions (peaks that show no other peak within ±4 cm⁻¹ to make up a unique set) at 3074, 1789 and 1398 cm⁻¹; and/or
(d) for the ball mill grind form (Form 2): has a differential scanning calorimetry spectrum having a first endotherm range of about 50° C. to about 88° C., and optionally a first endotherm at 66.49° C., and a second endotherm range of about 182° C. to about 230° C., and optionally a second endotherm at 195.4° C.; and/or
(e) for the lyophilization form (freeze drying, Form 1): has a differential scanning calorimetry spectrum having an endotherm range of about 175° C. to about 238° C., and optionally an endotherm at 201.4° C.

Methods by which the above referenced analyses were performed in order to identify these physical characteristics are described in the Examples.

The present invention relates to compositions comprising Compound I, wherein Compound I is present as Form A, Form B, Form C, Form D, Form E, Form F or the amorphous Form 1 or Form 2, as described below. It is noted that other crystalline and amorphous forms of Compound I may also be present in the composition.

In one variation, the composition comprises at least 0.25%, 0.5%, 1%, 5%, 10%, 25%, 50%, 75%, 80%, 85%, 90%, 95%, 97%, or 99% of Compound I where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F or the amorphous Form 1 or Form 2. The composition may optionally be a pharmaceutical composition. The pharmaceutical composition may optionally further include one or more pharmaceutical carriers.

Also provided are kits and other articles of manufacture comprising a composition that comprises Compound I, wherein Compound I is present as Form A, Form B, Form C, Form D, Form E, Form F or the amorphous Form 1 or Form 2. In one variation, the composition comprises at least 0.25%, 0.5%, 1%, 5%, 10%, 25%, 50%, 75%, 80%, 85%, 90%, 95%, 97%, or 99% of Compound I where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F or the amorphous Form 1 or Form 2. The composition in the kits and articles of manufacture may optionally be a pharmaceutical composition. The pharmaceutical composition may optionally further include one or more pharmaceutical carriers.

In regard to each of the above embodiments including a pharmaceutical composition, the pharmaceutical composition may be formulated in any manner where a portion of the compound is at least partially preserved in a given polymorphic form. Optionally, a portion of the compound is at least partially preserved in a given polymorphic form for a period of time subsequent to administration of the pharmaceutical formulation to a human.

8. Methods of Making Form A Through Form F and Amorphous Forms 1 and 2

Various methods are also provided for making Form A through Form F and amorphous Forms 1 and 2. Various methods are also provided for manufacturing pharmaceutical compositions, kits and other articles of manufacture comprising one or more of Form A through Form F and amorphous Form 1 and Form 2.

9. Methods of Using Form A Through Form F and Amorphous Forms 1 and 2

Methods of using a pharmaceutical composition, kit and other article of manufacture comprising one or more of Form A through Form F and amorphous Forms 1 and 2 to treat various diseases are also provided.

In one embodiment, the present invention relates to a method of inhibiting dipeptidyl peptidases comprising administering a composition where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F, amorphous Form 1 or amorphous Form 2. Optionally, the composition comprises at least 0.25%, 0.5%, 1%, 5%, 10%, 25%, 50%, 75%, 80%, 85%, 90%, 95%, 97%, or 99% of Compound I.

In another embodiment, the present invention relates to a method of inhibiting dipeptidyl peptidases in a subject (e.g., human body) with Compound I by administering Compound I where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F, amorphous Form 1 or amorphous Form 2 when the compound is administered. Optionally, the composition comprises at least 0.25%, 0.5%, 1%, 5%, 10%, 25%, 50%, 75%, 80%, 85%, 90%, 95%, 97%, or 99% of Compound I.

In another embodiment, the present invention relates to a method of inhibiting dipeptidyl peptidases in a subject (e.g., human body) with Compound I by administering Compound I where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F, amorphous Form 1 or amorphous Form 2 for a period of time after the compound has been administered to a human. Optionally, the composition comprises at least 0.25%, 0.5%, 1%, 5%, 10%, 25%, 50%, 75%, 80%, 85%, 90%, 95%, 97%, or 99% of Compound I.

In still another embodiment, the present invention provides a method of treating a disease state for which dipeptidyl peptidases possesses activity that contributes to the pathology and/or symptomology of the disease state, comprising administering to a subject (e.g., human body) a composition where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F, amorphous Form 1 or amorphous Form 2 when administered. Optionally, the composition comprises at least 0.25%, 0.5%, 1%, 5%, 10%, 25%, 50%, 75%, 80%, 85%, 90%, 95%, 97%, or 99% of Compound I.

In still another embodiment, the present invention provides a method of treating a disease state for which dipeptidyl peptidases possesses activity that contributes to the pathology and/or symptomology of the disease state, comprising causing a composition to be present in a subject (e.g., human body) where greater than 1%, 5%, 10%, 25%, 50%, 75%, 80%, 85%, 90%, 95%, 97% or 99% of Compound I (by weight) is present in the composition as Form A, Form B, Form C, Form D, Form E, Form F, amorphous Form 1 or amorphous Form 2 for a period of time after the composition has been administered to a human. Optionally, the composition comprises at least 0.25%, 0.5%, 1%, 5%, 10%, 25%, 50%, 75%, 80%, 85%, 90%, 95%, 97%, or 99% of Compound I.

In another embodiment, a method is provided for preventing, delaying the of progression, and/or treating conditions mediated by DPP-IV, in particular diabetes and more particularly, type 2 diabetes mellitus, diabetic dislipidemia, conditions of impaired glucose tolerance (IGT), conditions of impaired fasting plasma glucose (IFG), metabolic acidosis, ketosis, appetite regulation and obesity.

### BRIEF DESCRIPTION OF THE FIGURES

FIG. 1 illustrates the XRPD pattern of Form A, wherein the “XRPD pattern” is a plot of the intensity of diffracted lines.

FIG. 2 is a plot of TGA data and the DSC data for Form A.

FIG. 3 is a plot of the IR absorption spectrum for Form A.

FIG. 4 is a plot of the Raman absorption spectrum for Form A.

FIG. 5 is a plot of the proton NMR spectrum for Form A.

FIG. 6 illustrates the XRPD pattern of Form B, wherein the “XRPD pattern” is a plot of the intensity of diffracted lines.

FIG. 7 is a plot of TGA data and the DSC data for Form B.

FIG. 8 is a plot of the IR absorption spectrum for Form B.

FIG. 9 is a plot of the Raman absorption spectrum for Form B.

FIG. 10 is a plot of the proton NMR spectrum for Form B.

FIG. 11 illustrates the XRPD pattern of Form C, wherein the “XRPD pattern” is a plot of the intensity of diffracted lines.

FIG. 12 is a plot of TGA data and the DSC data for Form C.

FIG. 13 is a plot of the IR absorption spectrum for Form C.

FIG. 14 is a plot of the Raman absorption spectrum for Form C.

FIG. 15 is a plot of the proton NMR spectrum for Form C.

FIG. 16 illustrates the XRPD pattern of Form D, wherein the “XRPD pattern” is a plot of the intensity of diffracted lines.

FIG. 17 is a plot of TGA data for Form D.

FIG. 18 illustrates the XRPD pattern of Form E, wherein the “XRPD pattern” is a plot of the intensity of diffracted lines.

FIG. 19 is a plot of TGA data and the DSC data for Form E.

FIG. 20 is a plot of the IR absorption spectrum for Form E.

FIG. 21 is a plot of the Raman absorption spectrum for Form E.

FIG. 22 is a plot of the proton NMR spectrum for Form E.

FIG. 23 illustrates the XRPD pattern of Form F, wherein the “XRPD pattern” is a plot of the intensity of diffracted lines.

FIG. 24 illustrates the XRPD pattern of amorphous Form 1, wherein the “XRPD pattern” is a plot of the intensity of diffracted lines.

FIG. 25 is a plot of TGA data and the DSC data for amorphous Form 1.

FIG. 26 is a plot of the IR absorption spectrum for amorphous Form 1.

FIG. 27 illustrates the XRPD pattern of amorphous Form 2, wherein the “XRPD pattern” is a plot of the intensity of diffracted lines.

FIG. 28 is a plot of TGA data and the DSC data for amorphous Form 2.

FIG. 29 is a plot of the proton NMR spectrum for amorphous Form 2.

FIG. 30 is a plot of the IR absorption spectrum for amorphous Form 2.

FIG. 31 is a plot of the Raman absorption spectrum for amorphous Form 2.

### DETAILED DESCRIPTION OF THE INVENTION

The present invention provides novel polymorphs of Compound I, as well as compositions comprising Compound I where at least a portion of Compound I is present in the composition as Form A, Form B, Form C, Form D, Form E, Form F, amorphous Form 1 or amorphous Form 2.

Also provided are kits and other articles of manufacture with compositions comprising Compound I where at least a portion of Compound I is present in the composition as Form A, Form B, Form C, Form D, Form E, Form F, amorphous Form 1 or amorphous Form 2.

Various methods are also provided including methods of making the disclosed Form A, Form B, Form C, Form D, Form E, Form F, amorphous Form 1 and amorphous Form 2, methods for manufacturing pharmaceutical compositions comprising Compound I where at least a portion of Compound I is present in the composition as Form A, Form B, Form C, Form D, Form E, Form F, amorphous Form 1 and amorphous Form 2, and methods of using compositions comprising Compound I where at least a portion of Compound I is present in the composition as Form A, Form B, Form C, Form D, Form E, Form F, amorphous Form 1 or amorphous Form 2.

1. Preparation of Compound I

Various methods may be used to synthesize Compound I. Representative methods for synthesizing Compound I are provided in Example 1. It is noted, however, that other synthetic routes may also be used to synthesize Compound I including those disclosed in application Ser. No. 10/918,317, filed Aug. 12, 2004, which is hereby incorporated by reference in its entirety.

2. Preparation of Polymorphs

In general, a given polymorph of a compound may be obtained by direct crystallization of the compound or by crystallization of the compound followed by interconversion from another polymorphic form or from an amorphous state. The Examples describe methods for testing the solubility of Compound I as well as methods for screening for crystallization conditions for Compound I. The Examples also describe how Compound I, once solubilized, may be crystallized. Further, the Examples describe interconversion studies that were performed for Compound I.

Depending on the method by which a compound is crystallized, the resulting composition may contain different amounts of the compound in crystalline form as opposed to as an amorphous material. Also, the resulting composition may contain differing mixtures of different polymorphic forms of the compound.

“Crystalline”, as the term is used herein, refers to a material that contains a specific compound, which may be hydrated and/or solvated, and has sufficient crystalline content to exhibit a discernable diffraction pattern by XRPD or other diffraction techniques. Often, a crystalline material that is obtained by direct crystallization of a compound dissolved in a solution or interconversion of crystals obtained under different crystallization conditions, will have crystals that contain the solvent used in the crystallization, termed a crystalline solvate. Also, the specific solvent system and physical embodiment in which the crystallization is performed, collectively termed crystallization conditions, may result in the crystalline material having physical and chemical properties that are unique to the crystallization conditions, generally due to the orientation of the chemical moieties of the compound with respect to each other within the crystal and/or the predominance of a specific polymorphic form of the compound in the crystalline material.

Depending upon the polymorphic form(s) of the compound that are present in a composition, various amounts of the compound in an amorphous solid state may also be present, either as a side product of the initial crystallization, and/or a product of degradation of the crystals comprising the crystalline material. Thus, crystalline, as the term is used herein, contemplates that the composition may include amorphous content; the presence of the crystalline material among the amorphous material being detectably among other methods by the composition having a discernable diffraction pattern.

The amorphous content of a crystalline material may be increased by grinding or pulverizing the material, which is evidenced by broadening of diffraction and other spectral lines relative to the crystalline material prior to grinding. Sufficient grinding and/or pulverizing may broaden the lines relative to the crystalline material prior to grinding to the extent that the XRPD or other crystal specific spectrum may become undiscernable, making the material substantially amorphous or quasi-amorphous.

“Amorphous”, as the term is used herein, refers to a composition comprising a compound that contains too little crystalline content of the compound to yield a discernable pattern by XRPD or other diffraction techniques. Glassy materials are a type of amorphous material. Amorphous materials do not have a true crystal lattice, and are consequently glassy rather than true solids, technically resembling very viscous non-crystalline liquids. Rather than being true solids, glasses may better be described as quasi-solid amorphous material. Thus, an amorphous material refers to a quasi-solid, glassy material. Precipitation of a compound from solution, often affected by rapid evaporation of solvent, is known to favor the compound forming an amorphous solid as opposed to crystals. A compound in an amorphous state may be produced by rapidly evaporating solvent from a solvated compound, or by grinding, pulverizing or otherwise physically pressurizing or abrading the compound while in a crystalline state. General methods for precipitating and crystallizing a compound may be applied to prepare the various polymorphs described herein. These general methods are known to those skilled in the art of synthetic organic chemistry and pharmaceutical formulation, and are described, for example, by J. March, “Advanced Organic Chemistry: Reactions, Mechanisms and Structure,” 4^{th} Ed. (New York: Wiley-Interscience, 1992).

“Broad” or “broadened”, as the term is used herein to describe spectral lines, including XRPD, NMR and IR spectroscopy lines, is a relative term that relates to the line width of a baseline spectrum. The baseline spectrum is often that of an unmanipulated crystalline form of a specific compound as obtained directly from a given set of physical and chemical conditions, including solvent composition and properties such as temperature and pressure. For example, broadened can be used to describe the spectral lines of a XRPD spectrum of ground or pulverized material comprising a crystalline compound relative to the material prior to grinding. In materials where the constituent molecules, ions or atoms, as solvated or hydrated, are not tumbling rapidly, line broadening is indicative of increased randomness in the orientation of the chemical moieties of the compound, thus indicative of an increased amorphous content. When comparisons are made between crystalline materials obtained via different crystallization conditions, broader spectral lines indicate that the material producing the relatively broader spectral lines has a higher level of amorphous material.

Continued grinding would be expected to increase the amorphous content and further broaden the XRPD pattern with the limit of the XRPD pattern being so broadened that it can no longer be discerned above noise. When the XRPD pattern is broadened to the limit of being indiscernible, the material may be considered to no longer be a crystalline material, and instead be wholly amorphous. For material having increased amorphous content and wholly amorphous material, no peaks should be observed that would indicate grinding produces another form.

Compositions comprising a higher percentage of crystalline content (e.g., forming crystals having fewer lattice defects and proportionately less glassy material) are generally prepared when conditions are used that favor slower crystal formation, including those slowing solvent evaporation and those affecting kinetics. Crystallization conditions may be appropriately adjusted to obtain higher quality crystalline material as necessary. Thus, for example, if poor crystals are formed under an initial set of crystallization conditions, the solvent temperature may be reduced and ambient pressure above the solution may be increased relative to the initial set of crystallization conditions in order to slow crystallization.

As one will appreciate, depending on how a composition comprising a given compound is produced and then, once produced, how the composition is stored and manipulated, will influence the crystalline content of the composition. Accordingly, it is possible for a composition to comprise no crystalline content or may comprise higher concentrations of crystalline content.

It is further noted that a compound may be present in a given composition in one or more different polymorphic forms, as well as optionally also being present as an amorphous material. This may be the result of (a) physically mixing two or more different polymorphic forms; (b) having two or more different polymorphic forms be generated from crystallization conditions; (c) having all or a portion of a given polymorphic form convert into another polymorphic form; (d) having all or a portion of a compound in an amorphous state convert into two or more polymorphic forms; as well as for a host of other reasons.

As can be seen, depending on how a composition comprising a compound is prepared, the percentage, by weight, of that compound in a given polymorphic form can vary from 0% to 100%. According to the present invention, compositions are provided where greater than 1%, 5%, 10%, 25%, 50%, 75%, 80%, 85%, 90%, 95%, 97%, or 99% or more of Compound I (by weight) is present in the composition as Form A, Form B, Form C, Form D, Form E, Form F, amorphous Form 1 or amorphous Form 2.

3. Polymorphs of Compound I

Described herein are Form A, Form B, Form C, Form D, Form E, Form F, amorphous Form 1 and amorphous Form 2 of Compound I.

Various tests may be performed in order to physically characterize the crystalline state of Compound I including but not limited to X-ray powder diffraction (“XRPD”), differential scanning calorimetry (“DSC”), thermogravimetric analysis (“TGA”), hot stage microscopy, infrared spectrometry (“IR”), Raman spectrometry and Karl Fischer analysis. The Examples below describe the methods that were used to perform the various analyses reported herein. Where possible, the results of each test for each different polymorph are provided herein.

### A. Method of Making Form A of Compound I

The following describes procedures by which Form A polymorph of Compound I has been made from different samples of Compound I:

Sample No. 1888-46-01: Approximately 20 mg of Compound I was dissolved in a mixture of approximately 1.5 mL of acetone and 1.0 mL of water. The solution was filtered using a 0.2 μm nylon syringe filter into an open vial. The solution was allowed to evaporate to dryness at ambient conditions in a fume hood.

Sample No. 1888-48-03: Approximately 20 mg of Compound I was dissolved in 1.25 mL of methanol. The solution was filtered through a 0.2 μm nylon syringe filter. The filtered solution was allowed to evaporate in a fume hood at ambient temperature in a vial that was covered with a piece of aluminum foil containing pinholes. Solid material was collected by filtration before complete evaporation of the solvent.

Sample No. 1888-51-02: Approximately 20 mg of Compound I was dissolved in 1.0 mL of hot methanol. The solution was filtered using a 0.2 μm nylon syringe filter into a 1 dram vial. 3.0 mL of cold acetone was added and the solution was left at room temperature for approximately 24 hours. The solids were collected via vacuum filtration.

Sample No. 1888-51-03: Approximately 20 mg of Compound I was dissolved in 1.0 mL of hot methanol. The solution was filtered using a 0.2 μm nylon syringe filter into a 1 dr vial. 3.0 mL of cold toluene was added and the solution was left at room temperature for approximately 24 hours. The solids were collected via vacuum filtration.

Sample No. 1888-93-01: Approximately 93 mg of Compound I was dissolved in 1.0 mL of hot water (approximately 80° C.). The solution was filtered using a 0.2 μm nylon syringe filter into a 1 dr vial and allowed to cool slowly. The solids were collected via vacuum filtration.

### B. Characterization of Form A of Compound I

FIG. 1 illustrates the XRPD pattern of Form A. Major diffraction lines are observed for °2θ at approximately: 6.22, 7.92, 8.51, 12.77, 14.72, 15.48, 29.23 and 32.44. The XRPD pattern tends to indicate that confirmed that the material is a crystalline phase, which was designated form A.

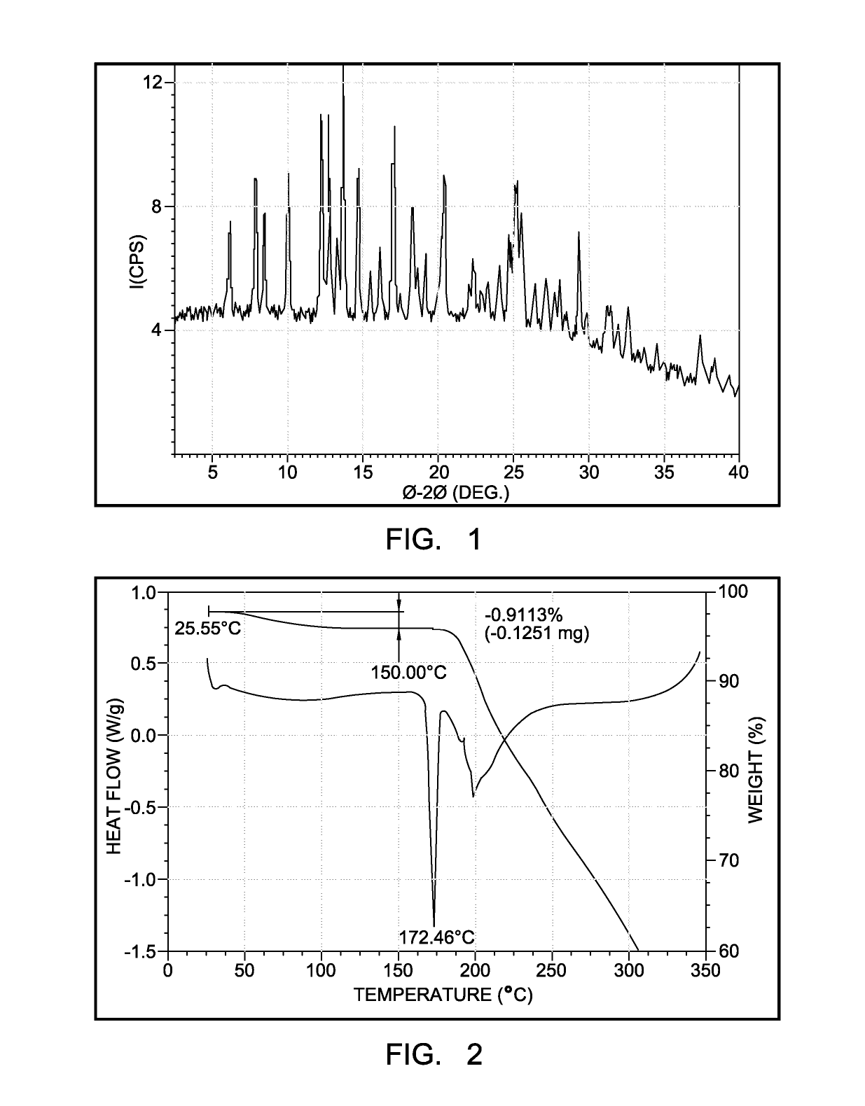

Thermogravimetric and DSC data for Form A is summarized below in Table 1A and plotted in FIG. 2. As can be seen in FIG. 2, the DSC curve exhibits several endothermic events. The endotherm maximum for the most predominant event is located near 172° C. A melting point experiment (sample no. 1888-62-01) confirmed that this endothermic event is associated with the melt of the material. The series of endothermic events above the melt endotherm were not characterized further, but likely correspond to the decomposition of the sample. A broad endothermic event located below the melt endotherm for the sample can also be seen in the DSC plot for the sample of form A. This event occurs in the same temperature region as the corresponding mass loss observed in the TGA plot for a sample of form A and is consistent with the loss of volatile material from the sample.

TABLE 1A
Thermal Data for Form A

| DSC Results¹ | TGA Results² |
|---|---|
| Endotherm at 172.5° C. | 0.9% at 150° C. |

¹Maximum temperature reported for transition
²Percent weight change from 35° C. to 200° C.

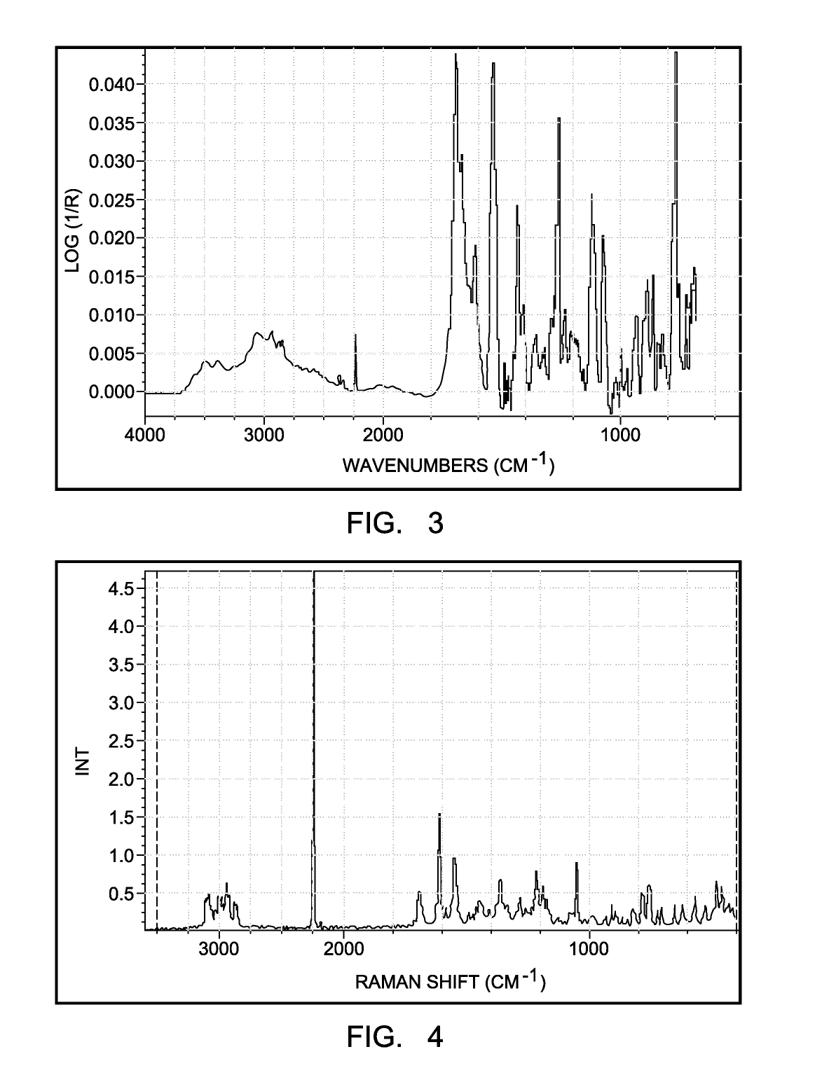

The IR spectrum for Form A (sample no. 1888-41-01) is plotted in FIG. 3. In one variation, IR peak positions that show no other peak within ±4 cm⁻¹ make up a unique set of peaks that can be used to identify the polymorph of Compound I.

The Raman spectrum for Form A (sample no. 1888-41-01) is provided in FIG. 4. In one variation, Raman peak positions that show no other peak within ±4 cm⁻¹ make up a unique set of peaks that can be used to identify the polymorph of Compound I.

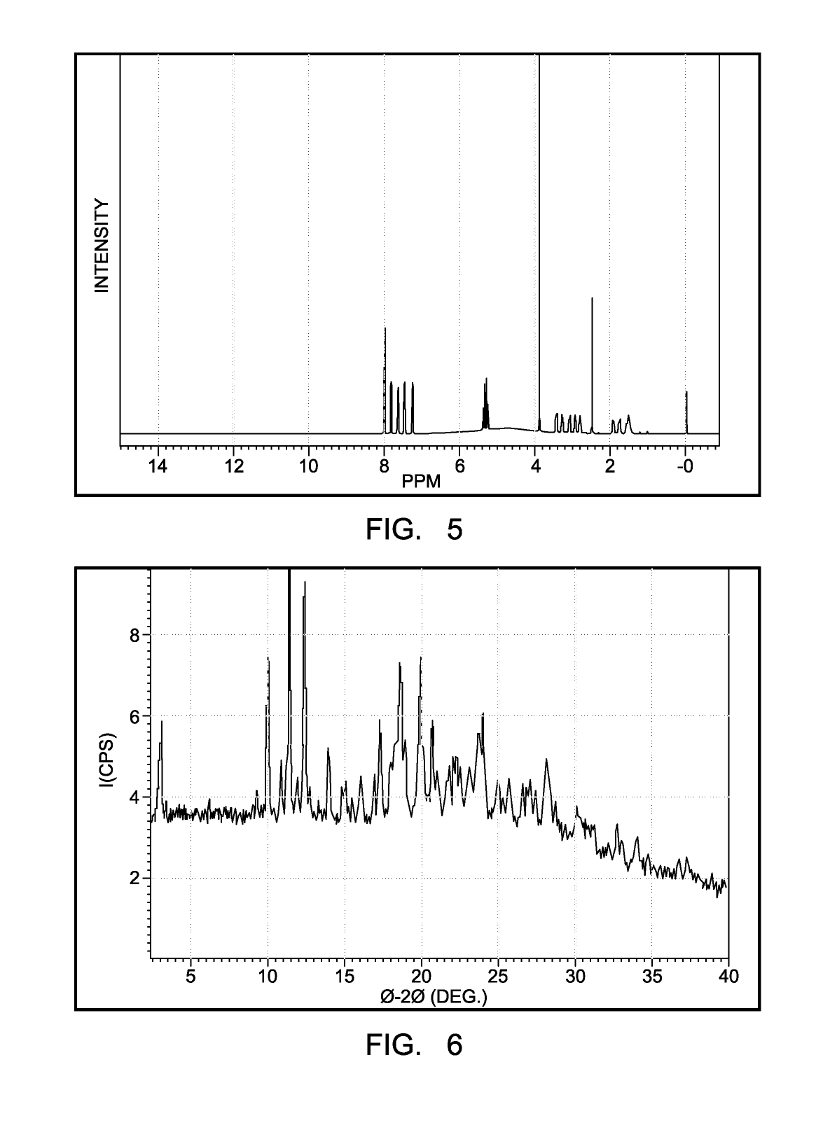

The NMR spectrum of Form A (FIG. 5) contains resonances that are attributable to Compound I, which indicates that the sample does not correspond to a decomposition product.

Data regarding the moisture sorption/desorption properties of Form A are summarized in Table 2A below. The analysis showed an initial weight loss of approximately 1.4% from ambient conditions to 5% RH and a weight gain of about 4.00% between 5% and 95% RH. The desorption cycle follows a similar path as the sorption cycle, where the mass gained during the sorption cycle is lost upon desorption in similar amounts at the various RH intervals. The weight change value for the desorption cycle can be used along with the molecular weight of Compound I to calculate that approximately 1 molecule of water is present for every molecule of compound at 95% RH. This calculation assumes that no water is present in the sample at the 5% RH step at the end of the analysis. The post moisture sorption/desorption sample was identified as form A by XRPD. In separate experiments form A was stressed under various RH conditions at room temperature (Table 3A). After several weeks none of these samples had undergone a phase change. This is evident in the respective XRPD patterns for the samples, which all correspond to form A. The gradual mass increase/decrease observed throughout the range of RH levels examined in the moisture sorption/desorption profile coupled with the lack of a phase change for form A upon RH stress indicates that this solid phase may contain a variable amount of water that will be dependent on the RH of the environment. This type of hydrated phase is referred to as a variable or non-stoichiometric hydrate.

TABLE 2A
Moisture Sorption/Desorption Data for Form A

| Elapsed | | | | |
|---|---|---|---|---|
| Time | Weight | Weight Change | Sample Temperature | Sample RH |
| (min) | (mg) | (%) | (° C.) | (%) |
| 0.1 | 7.536 | 0.00 | 24.98 | 10.82 |
| 41.6 | 7.427 | −1.44 | 24.99 | 5.18 |
| 213.9 | 7.724 | 4.00 | 24.99 | 94.77 |
| 391.0 | 7.433 | −3.77 | 24.97 | 4.99 |

TABLE 3A
Compound I Polymorph Screen - Solids-Based Experiments

| | | |
|---|---|---|
| Starting | | XRPD |
| Solid Phase | Conditions | Pattern |

| | |
|---|---|
| | Amorphous |
| | Amorphous |
| Cool of melt | Amorphous |
| Cool of melt (under vacuum) | Amorphous |
| 58% RH stress, ambient, 21 d | Form A |
| 75% RH stress, ambient, 21 d | Form A |
| 84% RH stress, ambient, 21 d | Form A |
| TGA to 150° C. & recovered | Form A |
| 6% RH stress, ambient, 21 d | Amorphous |
| 33% RH stress, ambient, 21 d | Amorphous |
| 58% RH stress, ambient, 21 d | Amorphous |
| 75% RH stress, ambient, 21 d | Form B |
| 84% RH stress, ambient, 21 d | Pattern F |

The results from a Karl Fischer water analysis performed on Form A was approximately 2.9 (±0.2) % water by weight. This value is slightly larger than the weight loss of ˜0.9% observed in the TGA plot for the sample from ambient temperature to 150° C. This discrepancy is possibly due to the sample losing mass upon temperature equilibration of the furnace prior to the start of the TGA temperature ramp.

### C. Method of Making Form B of Compound I

The following describes procedures by which Form B polymorph of Compound I has been made from different samples of Compound I:

Sample No. 1888-46-05: Approximately 20 mg of Compound I was dissolved in a mixture of 1.0 mL of tetrahydrofuran and 0.5 mL of water. The solution was filtered using a 0.2 μm nylon syringe filter into an open vial. The solution was allowed to evaporate to dryness at ambient conditions in a fume hood.

Sample No. 1888-66-04: Approximately 8 mg of amorphous material prepared by freeze-drying was weighed into an open vial. The vial was placed in a chamber containing a saturated salt solution at approximately 75% relative humidity [See “Standard Practice for Maintaining Constant Relative Humidity by Means of Aqueous Solutions” ASTM Standard E 104-02 (2002)]. The chamber was sealed and allowed to stand at ambient temperature for several weeks. Samples were analyzed by X-ray powder diffraction (XRPD) immediately after removing the sample from the RH chamber. The RH values for these salt solutions were obtained from an ASTM standard.

Sample No. 1888-84-02: Approximately 50 mg of Compound I was dissolved in a mixture of 2.0 mL dioxane and 1.0 mL of water. The solution was filtered using a 0.2 μm nylon syringe filter into an open vial. The solution was allowed to evaporate to dryness at ambient conditions in a fume hood.

Sample No. 1982-09-01: Approximately 79 mg of Compound I was dissolved in a mixture of 2.0 mL acetonitrile and 1.5 mL of water. The solution was filtered using a 0.2 μm nylon syringe filter into an open 20 mL vial. The solution was allowed to evaporate to dryness at ambient conditions in a fume hood.

### D. Characterization of Form B of Compound I

FIG. 6 illustrates the XRPD pattern of Form B. Major diffraction lines are observed at approximately at °2θ: 3.11, 10.96, 14.07, 20.04 and 20.82.

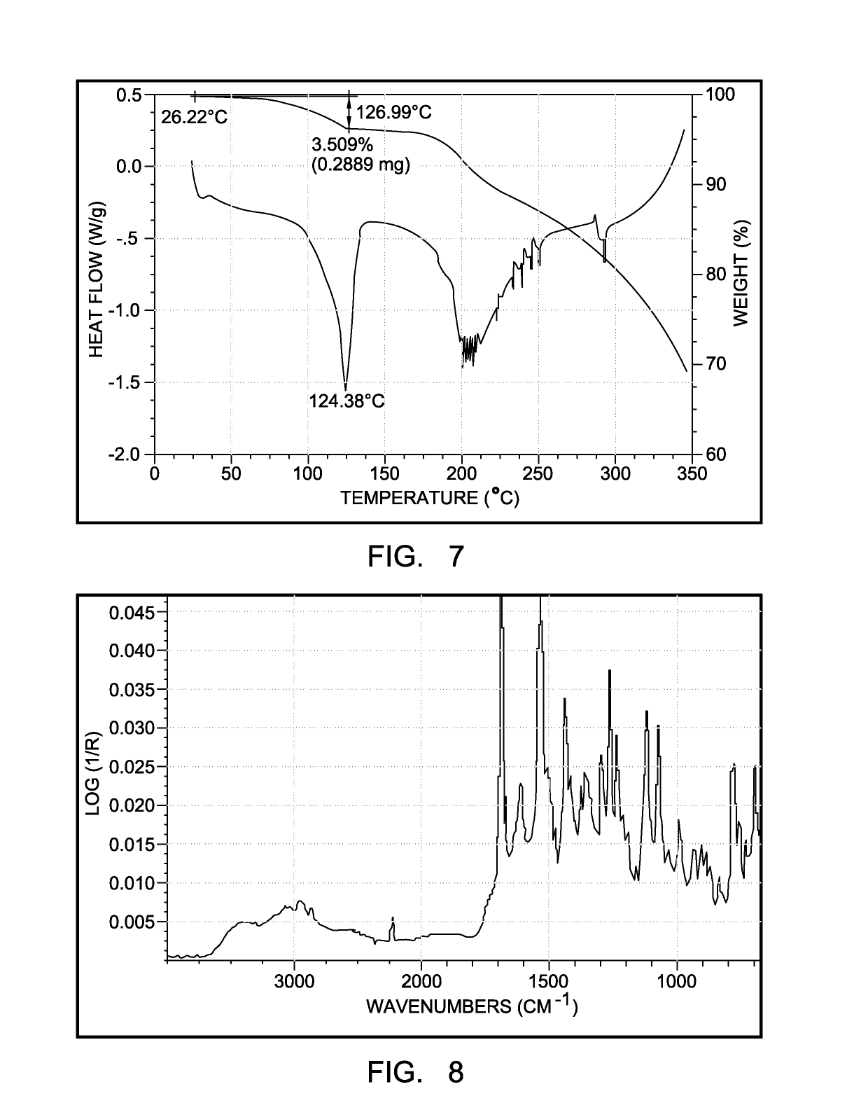

Thermogravimetric and DSC data for Form B is summarized below in Table 1B and plotted in FIG. 7. As can be seen in FIG. 7, DSC analysis shows two endothermic events. The first endothermic event occurred at ˜124° C., and is related to the ˜3.5% mass loss event observed in the TGA plot for the sample below 127° C. Assuming this weight loss is due to water, a loss of 3.5% would correspond to a molar ratio of about 1 molecule of water for each molecule of Compound I. The second endothermic event in the DSC plot occurred at higher temperatures and is likely due to decomposition of the sample. Significant weight loss occurred after 175° C. in the TGA plot, which is also consistent with decomposition.

TABLE 1B
Thermal Data for Form B

| DSC Results¹ | TGA Results² |
|---|---|
| Endotherm at 124.4° C. | 3.5% at 127° C. |

¹Maximum temperature reported for transition
²Percent weight change from 35° C. to 200° C.

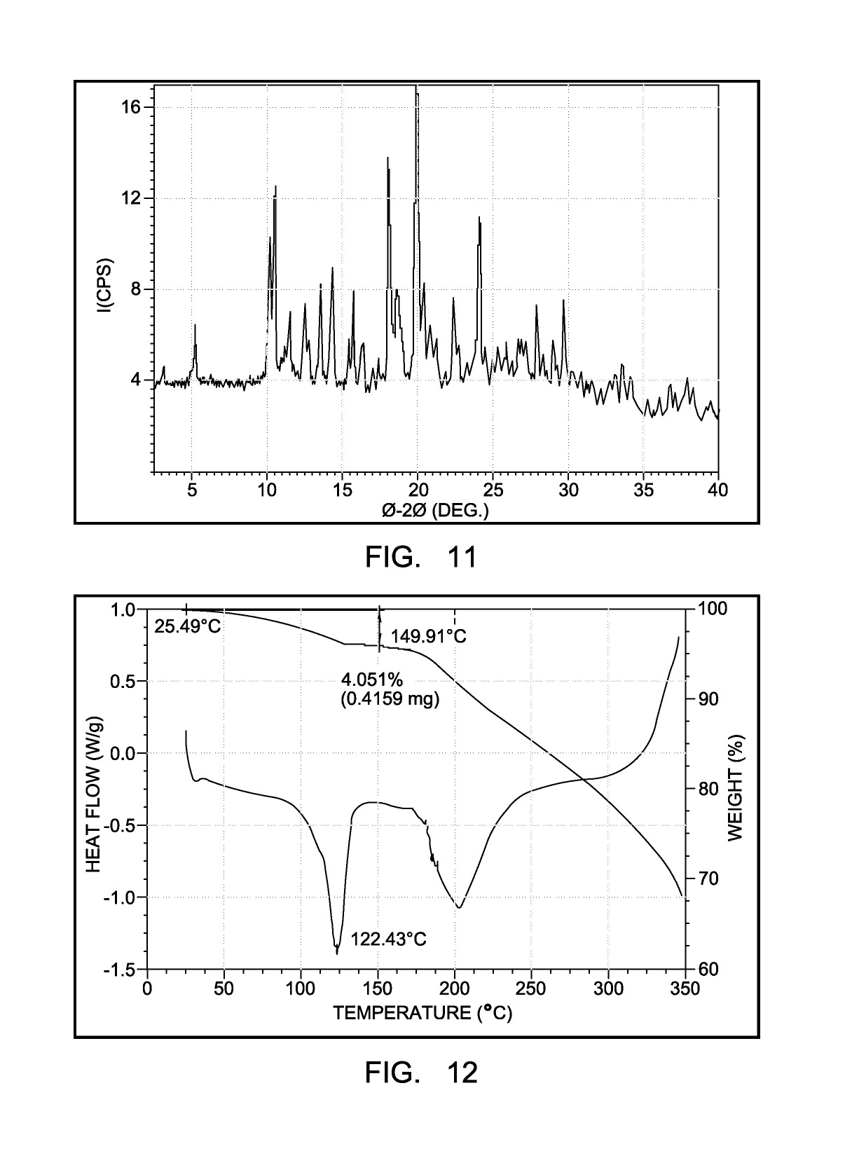

The NMR spectrum of Form B (FIG. 10) contains resonances that are attributable to Compound I, which indicates that the sample does not correspond to a decomposition product.

The IR spectrum for Form B is plotted in FIG. 8. In one variation, IR peak positions that show no other peak within ±4 cm⁻¹ make up a unique set of peaks that can be used to identify the polymorph of Compound I.

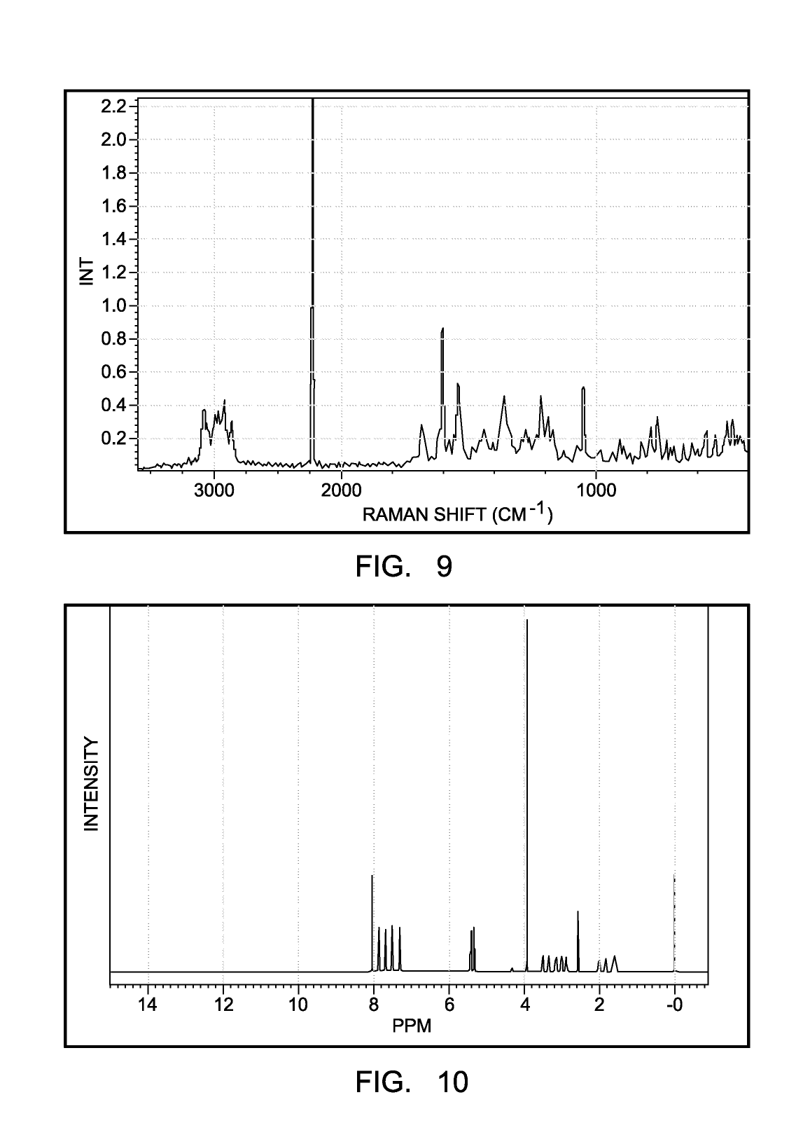

The Raman spectrum for Form B is provided in FIG. 9. In one variation, Raman peak positions that show no other peak within ±4 cm⁻¹ make up a unique set of peaks that can be used to identify the polymorph of Compound I.

Data regarding the moisture sorption/desorption properties of Form B are summarized in Table 2B below. The moisture sorption/desorption data show an initial weight loss of approximately 0.5% from ambient conditions to 5% RH and a 6.6% weight gain between 5% and 95% RH. This weight gain is lost upon desorption to provide a sample with roughly the same mass at 5% RH at the end of the experiment as at the beginning. Form B was not stable under the conditions of this experiment as the sample isolated after the experiment was identified as form C by XRPD analysis.

TABLE 2B
Moisture Sorption/Desorption Data for Form B

| Elapsed | | | | |
|---|---|---|---|---|
| Time | Weight | Weight Change | Sample Temperature | Sample RH |
| (min) | (mg) | (%) | (° C.) | (%) |
| 0.1 | 5.144 | 0.00 | 25.10 | 6.59 |
| 185.6 | 5.118 | −0.50 | 25.12 | 5.23 |
| 1848.8 | 5.458 | 6.64 | 25.07 | 94.66 |
| 3509.6 | 5.114 | −6.30 | 25.10 | 5.01 |

### E. Method of Making Form C of Compound I

The following describes procedures by which Form C polymorph of Compound I has been made from different samples of Compound I:

Sample No. 1888-46-06: Approximately 20 mg of Compound I02 was dissolved in a mixture of 1.0 mL ethanol and 1.0 mL of water. The solution was filtered using a 0.2 μm nylon syringe filter into an open vial. The solution was allowed to evaporate to dryness at ambient conditions in a fume hood.

Sample No. 1982-11-01: Approximately 50 mg of Compound I was dissolved in a mixture of 1.5 mL isopropanol and 1.5 mL of water. The solution was filtered using a 0.2 μm nylon syringe filter into an open vial. The solution was allowed to evaporate to dryness at ambient conditions in a fume hood.

### F. Characterization of Form C of Compound I

FIG. 11 illustrates the XRPD pattern of Form C. Major diffraction lines are observed at approximately at °2θ, 5.22, 14.29, 15.68, 18.04 and 29.58.

Thermogravimetric and DSC data for Form C is summarized below in Table 1C and plotted in FIG. 12. As can be seen in FIG. 12, DSC analysis showed two endothermic events. The first endothermic event occurred at ˜125° C., which is related to the ˜4.1% mass loss below 125° C. observed in the TGA plot for this sample. Assuming that this weight loss is due to water, a loss of 4.1% would correspond to a molar ratio of about 1.1 molecules of water for each molecule of Compound I. The second endothermic event in the DSC plot occurs at higher temperatures and is likely due to decomposition of the sample. Significant weight loss occurs after 175° C. in the TGA plot, which is also consistent with decomposition.

TABLE 1C
Thermal Data for Form C

| DSC Results¹ | TGA Results² |
|---|---|
| Endotherm at 122.4° C. | 4.1% at 150° C. |

¹Maximum temperature reported for transition
²Percent weight change from 35° C. to 200° C.

The NMR spectrum of Form C (FIG. 15) contains resonances that are attributable to Compound I, which indicates that the sample does not correspond to a decomposition product.

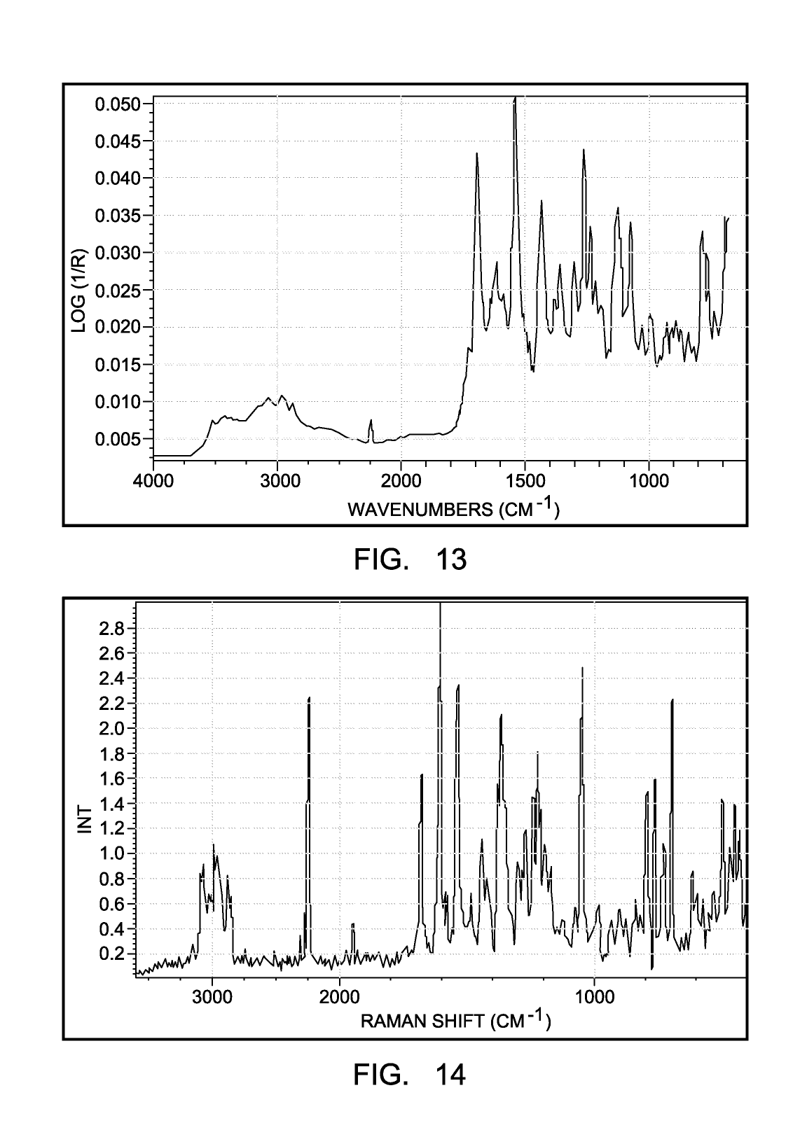

The IR spectrum for Form C is plotted in FIG. 13. In one variation, IR peak positions that show no other peak within ±4 cm⁻¹ make up a unique set of peaks that can be used to identify the polymorph of Compound I.

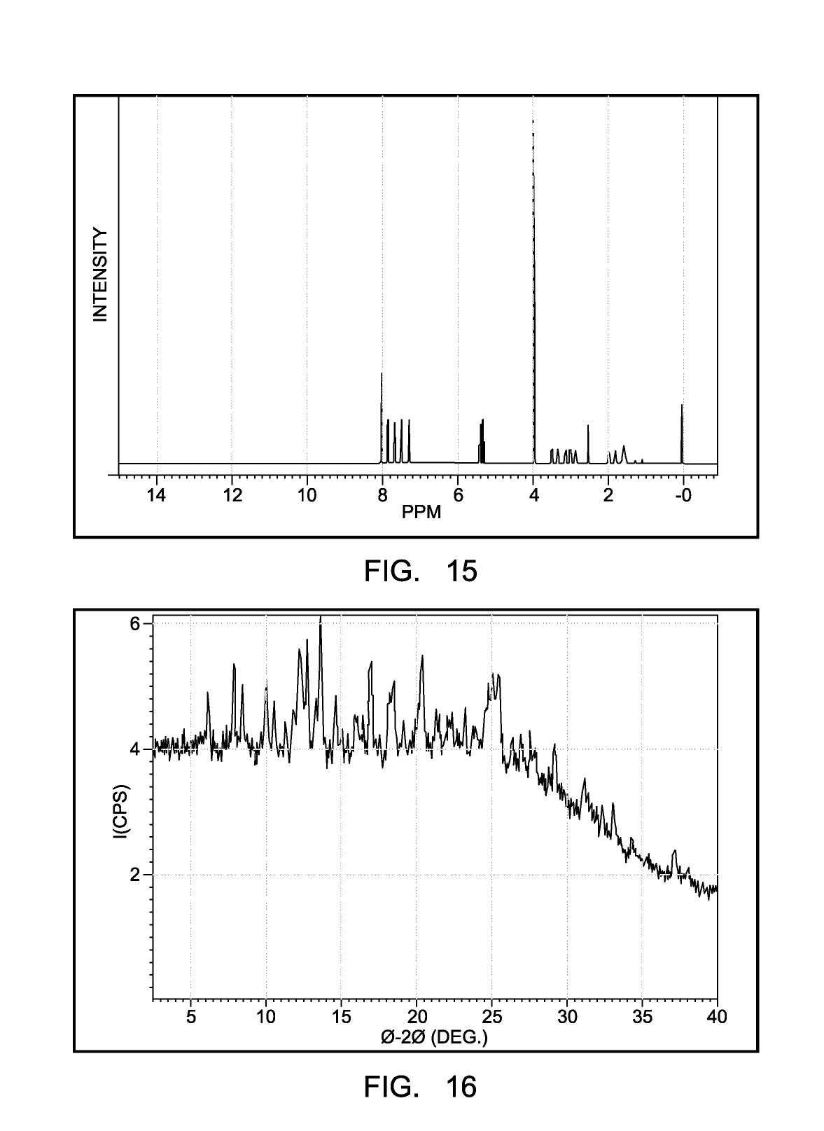

The Raman spectrum for Form C (sample no. 1982-11-01) is provided in FIG. 14. In one variation, Raman peak positions that show no other peak within ±4 cm⁻¹ make up a unique set of peaks that can be used to identify the polymorph of Compound I.

Data regarding the moisture sorption/desorption properties of Form C are summarized in Table 2C below. The moisture sorption/desorption data show an initial weight loss of approximately 0.9% from ambient conditions to 5% RH and a 5.7% weight gain between 5% and 95% RH, this weight gain is lost upon desorption. The desorption cycle follows a similar path as the sorption cycle. The weight change value for the desorption cycle can be used along with the molecular weight of Compound I to calculate that approximately 1.5 molecules of water is present for every molecule of compound at 95% RH. The post moisture sorption/desorption sample was identified as form C by XRPD.

TABLE 2C
Moisture Sorption/Desorption Data for Form C

| Elapsed | | | | |
|---|---|---|---|---|
| Time | Weight | Weight Change | Sample Temperature | Sample RH |
| (min) | (mg) | (%) | (° C.) | (%) |
| 0.1 | 6.498 | 0.00 | 24.96 | 2.20 |
| 156.2 | 6.442 | −0.873 | 24.97 | 5.1 |
| 656.6 | 6.807 | 4.744 | 24.97 | 94.71 |
| 1273.9 | 6.440 | −0.899 | 25.00 | 4.98 |

### G. Method of Making Form D of Compound I

The following describes procedures by which Form D polymorph of Compound I has been made from different samples of Compound I:

Sample No. 1888-50-03: Approximately 96 mg of Compound I was dissolved in 1.0 mL of hot water (approximately 80° C.). The solution was filtered using a 0.2 μm nylon syringe filter into a 1 dr vial. The vial was placed in an ice bath; solids were collected via vacuum filtration.

Sample No. 1888-71-02: Approximately 4 mg of amorphous material prepared by freeze-drying was weighed into an open vial. The vial was placed in a chamber containing methanol; the chamber was sealed and allowed to stand at ambient temperature for several weeks. Samples were analyzed by X-ray powder diffraction (XRPD) immediately after removing the sample from the chamber.

### H. Characterization of Form D of Compound I

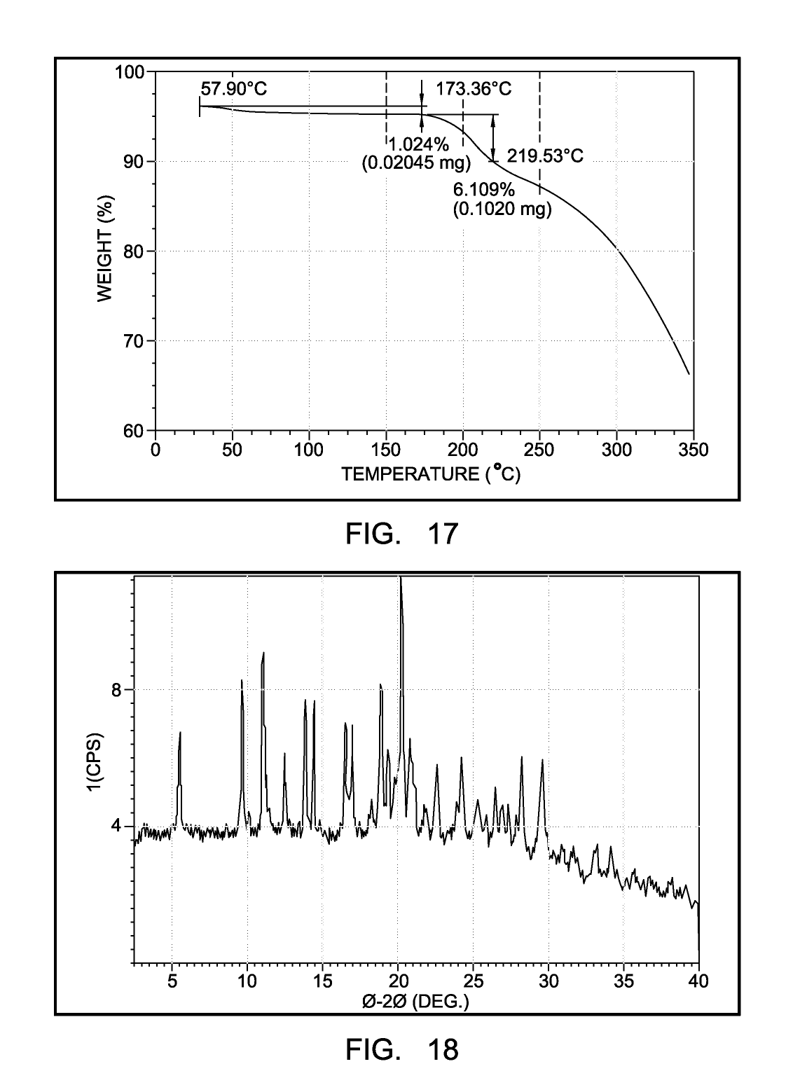

FIG. 16 illustrates the XRPD pattern of Form D. Major diffraction lines are observed at approximately at °2θ: 6.21, 7.90, 8.50 and 10.58.

Thermogravimetric and DSC data for Form D is summarized below in Table 1D and plotted in FIG. 17. As can be seen in FIG. 17, the thermogravimetric data shows a gradual weight loss of approximately 1.0% between 26° C. and 173° C. Significant weight loss occurred after 175° C., which is consistent with decomposition.

TABLE 1D
Thermal Data for Form D
TGA Results¹
1.0% at 173.3° C.
¹Percent weight change from 35° C. to 200° C.

### I. Method of Making Form E of Compound I

The following describes procedures by which Form E polymorph of Compound I has been made from different samples of Compound I:

Sample No. 1982-05-01: Approximately 20 mg of Compound I was dissolved in 1.0 mL of 16% water in acetonitrile at approximately 50° C. The solution was filtered using a 0.2 μm nylon syringe filter into a warm 1 dr vial. The solution was allowed to evaporate to dryness at ambient conditions in a fume hood.

Sample No. 1982-09-02: Approximately 77 mg of Compound I was dissolved in a mixture of 3.0 mL of dioxane and 1.5 mL of water. The solution was filtered using a 0.2 μm nylon syringe filter into a 20 mL vial. The solution was allowed to evaporate to dryness at ambient conditions in a fume hood.

### J. Characterization of Form E of Compound I

FIG. 18 illustrates the XRPD pattern of Form E. Major diffraction lines are observed at approximately at °2θ: 5.51, 9.66, 11.03, 13.83, 14.41, 16.50, 18.83, 22.51, 28.15 and 29.47.

Thermogravimetric and DSC data for Form E is summarized below in Table 1E and plotted in FIG. 19. DSC analysis showed two endothermic events. The first endothermic event occurred at ˜122° C., the second has an onset temperature of ˜200° C. The TG curve shows a weight loss of approximately 3.0% at ˜150° C. Assuming this weight loss is due to water, a loss of 3.0% would correspond to a molar ratio of about 0.8 molecules of water for each molecule of Compound I.

TABLE 1E
Thermal Data for Form E

| DSC Results¹ | TGA Results² |
|---|---|
| Endotherm 121.9° C. | 3.0% at 150° C. |

¹Maximum temperature reported for transition
²Percent weight change from 35° C. to 200° C.

The NMR spectrum of Form E (FIG. 22) contains resonances that are attributable to Compound I, which indicates that the sample does not correspond to a decomposition product.

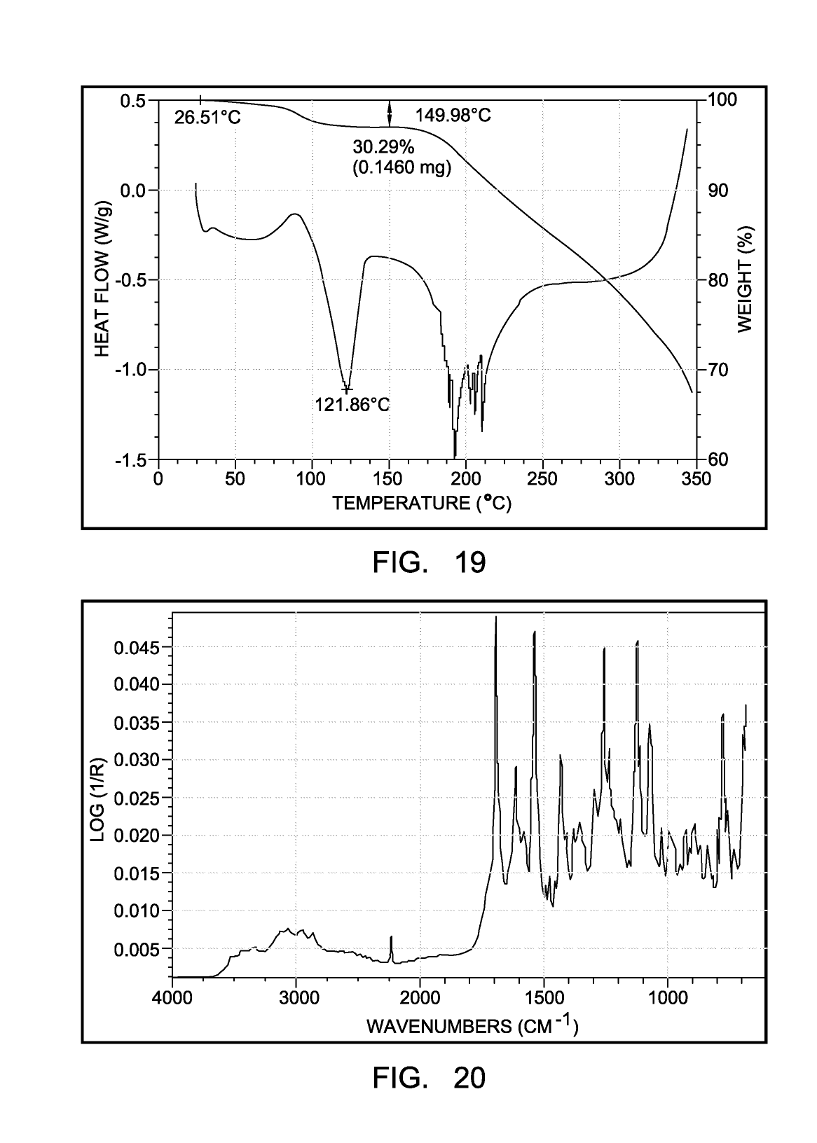

The IR spectrum for Form E (sample no. 1982-09-02) is plotted in FIG. 20. In one variation, IR peak positions that show no other peak within ±4 cm⁻¹ make up a unique set of peaks that can be used to identify the polymorph of Compound I.

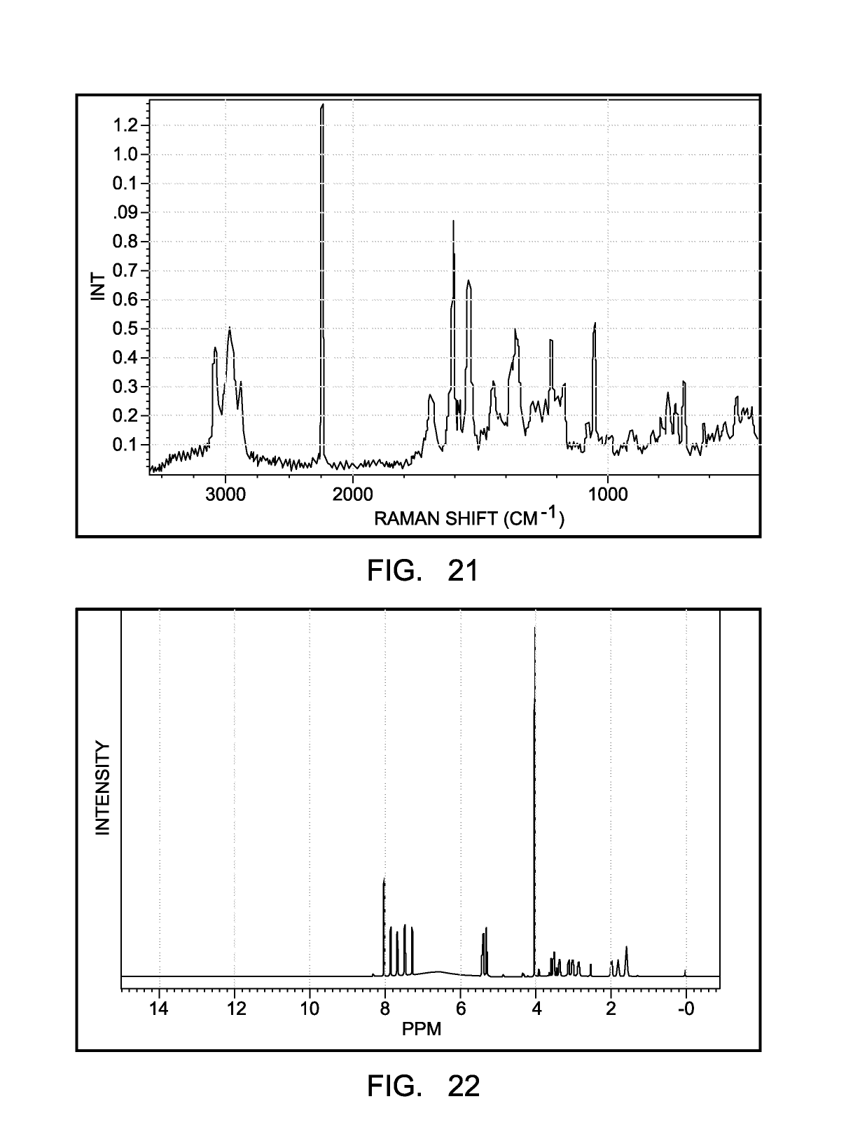

The Raman spectrum for Form E (sample no. 1982-09-02) is provided in FIG. 21. In one variation, Raman peak positions that show no other peak within ±4 cm⁻¹ make up a unique set of peaks that can be used to identify the polymorph of Compound I.

Data regarding the moisture sorption/desorption properties of Form E are summarized in Table 2D below. The moisture sorption/desorption profile shows an initial weight loss of approximately 0.4% from ambient conditions to 5% RH and an additional 9.2% weight gain between 5% and 95% RH, and this weight gain is lost upon desorption, similar path as the sorption cycle. The post moisture sorption/desorption sample was identified as form C by XRPD.

TABLE 2D
Moisture Sorption/Desorption Data for Form E

| Elapsed Time | Weight | Weight | Sample Temperature | Sample |
|---|---|---|---|---|
| (min) | (mg) | Change (%) | (° C.) | RH (%) |
| 0.1 | 5.356 | 0.00 | 25.20 | 11.45 |
| 43.7 | 5.337 | −0.352 | 25.21 | 5.21 |
| 363.2 | 5.827 | 8.801 | 25.21 | 95.08 |
| 693.4 | 5.361 | 0.098 | 25.22 | 4.73 |

### K. Method of Making Form F of Compound I

The following describes procedures by which Form F polymorph of Compound I has been made from different samples of Compound I:

Sample No. 1888-66-05: Approximately 8 mg of amorphous material prepared by freeze-drying was weighed into an open vial. The vial was placed in a chamber containing a saturated salt solution at approximately 84% relative humidity [“Standard Practice for Maintaining Constant Relative Humidity by Means of Aqueous Solutions” ASTM Standard E 104-02 (2002)]. The chamber was sealed and allowed to stand at ambient temperature for several weeks. The sample was analyzed by X-ray powder diffraction (XRPD) immediately upon removal from the RH chamber.

### L. Characterization of Form F of Compound I

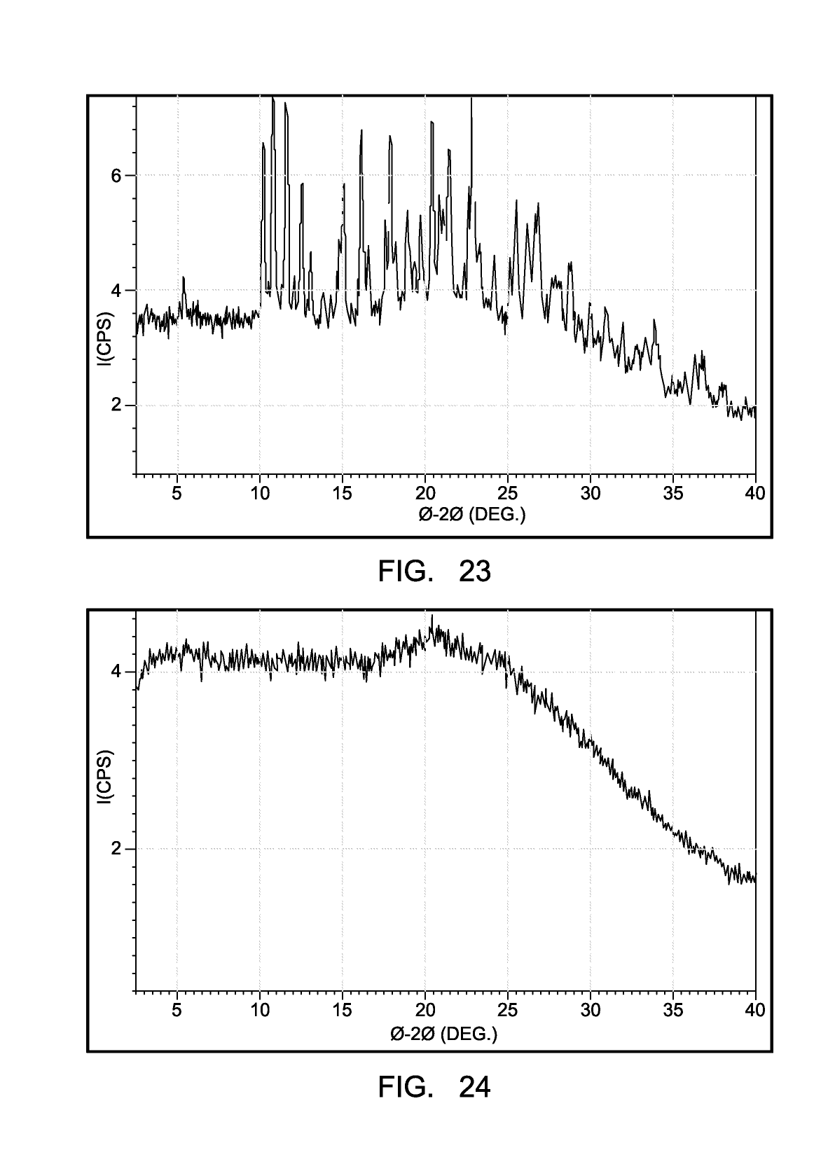

FIG. 23 illustrates the XRPD pattern of Form F. Major diffraction lines are observed for °2θ at approximately: 10.79, 11.59, 12.51 and 13.02.

### M. Method of Making Amorphous Form 1 and Form 2 of Compound I

Amorphous Form 1 polymorph of Compound I has been prepared as follows. Approximately 117 mg of Compound I was dissolved in 2.0 mL of water. This solution was filtered through 0.2 μm nylon syringe filter into a 50 ml round bottom flask. The solution was frozen in a dry ice/acetone bath and placed on a commercial freeze dryer equipped with a rotary vane mechanical vacuum pump. The flask was wrapped in glass wool and aluminum foil to insulate the frozen solution during the freeze drying operation.

Amorphous Form 2 polymorph of Compound I may be prepared by milling approximately 38 g of Compound I using a ball mill for 20 minutes at a frequency of 30 Hz.

### N. Characterization of Amorphous Form 1 and Form 2 of Compound I

Polymorph Amorphous Form 1:

FIG. 24 illustrates the XRPD pattern of amorphous Form 1 (Sample No. 1888-41-03). The XRPD data show poor signal-to-noise ratio. The XRPD pattern tends to indicate that Form 1 is an amorphous form of Compound I.

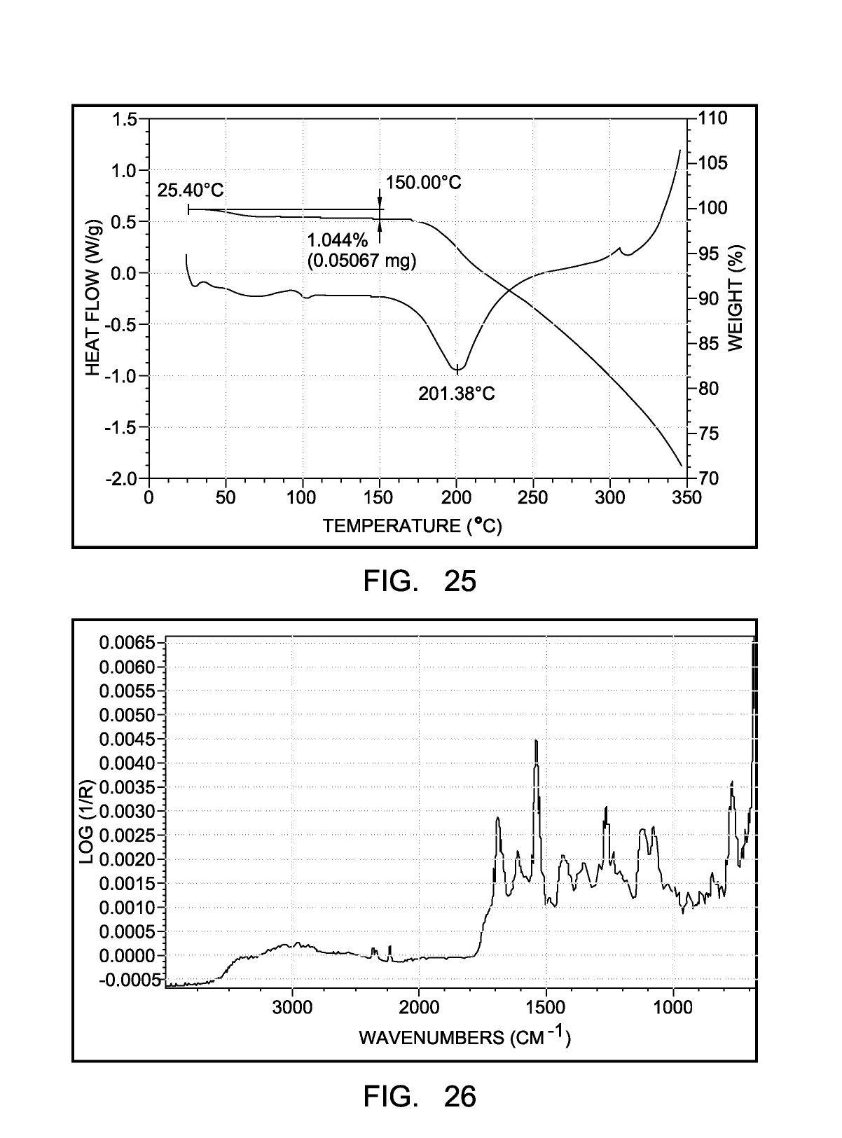

Thermogravimetric and DSC data for amorphous Form 1 are summarized below in Table 1F and plotted in FIG. 25. As can be seen in FIG. 25, volatile material is present in the sample as a mass loss of about 1.6% is observed in the analysis from 26° C. to 150° C. Significant weight loss occurred after 175° C., which is consistent with decomposition. Thermal analysis of a sample of amorphous solids prepared by lyophilization provided similar results to the milled material. In order to determine the glass transition temperature (Tg) for amorphous material cyclic DSC experiments were conducted. In one experiment amorphous material was generated in situ by cooling the sample in the DSC pan after the melt of Form A. This procedure proved to be unsatisfactory due to the poorly defined features of the plot near the Tg upon temperature cycling. Another experiment involved DSC temperature cycling of a sample of amorphous material prepared by lyophilization. Temperature cycling allows for the removal of moisture from the solids, the presence of which would affect the Tg value. Analysis of the data provides a Tg value (inflection point) of approximately 98.9° C.

TABLE 1F
Thermal Data for Amorphous Form 1

| DSC Results¹ | TGA Results² |
|---|---|
| Endotherms 201.4 | 1.1% at 150° C. |

¹Maximum temperature reported for transition
²Percent weight change from 35° C. to 200° C.

The IR spectrum for amorphous Form 1 (sample no. 1888-41-03) is plotted in FIG. 26. In one variation, IR peak positions that show no other peak within ±4 cm⁻¹ make up a unique set of peaks that can be used to identify the polymorph of Compound I.

Data regarding the moisture sorption/desorption properties of amorphous Form 1 are summarized in Table 2E below. The moisture sorption/desorption data show an initial weight gain of approximately 0.2% from ambient conditions to 5% RH and an additional weight gain of 5.8% between 5% and 95% RH. Between 75% and 85% RH the sample lost weight, which is suggestive of a crystallization event. The sample recovered after the moisture sorption/desorption experiment was tentatively identified as containing form A.

TABLE 2E
Moisture Sorption/Desorption Data for Amorphous Form 1

| | | | Sample | |
|---|---|---|---|---|
| Elapsed Time | Weight | Weight Change | Temperature | Sample RH |
| (min) | (mg) | (%) | (° C.) | (%) |
| 0.1 | 3.210 | 0.00 | 24.81 | 5.24 |
| 29.6 | 3.208 | −0.054 | 24.81 | 5.53 |
| 680.7 | 3.640 | 13.393 | 24.80 | 95.05 |
| 1647.8 | 3.238 | 0.868 | 24.79 | 5.09 |

The poor signal-to-noise ratio of the XRPD pattern indicates that amorphous material is likely still present to some extent in this sample. Contrary to the moisture balance results obtained for the milled material, freeze dried Compound I exhibited a markedly different moisture sorption/desorption profile. The overall weight gain from 5% to 95% RH was on the order of 13.5%. In addition, a possible crystallization event took place between 85% and 95% RH.

Polymorph Form 2:

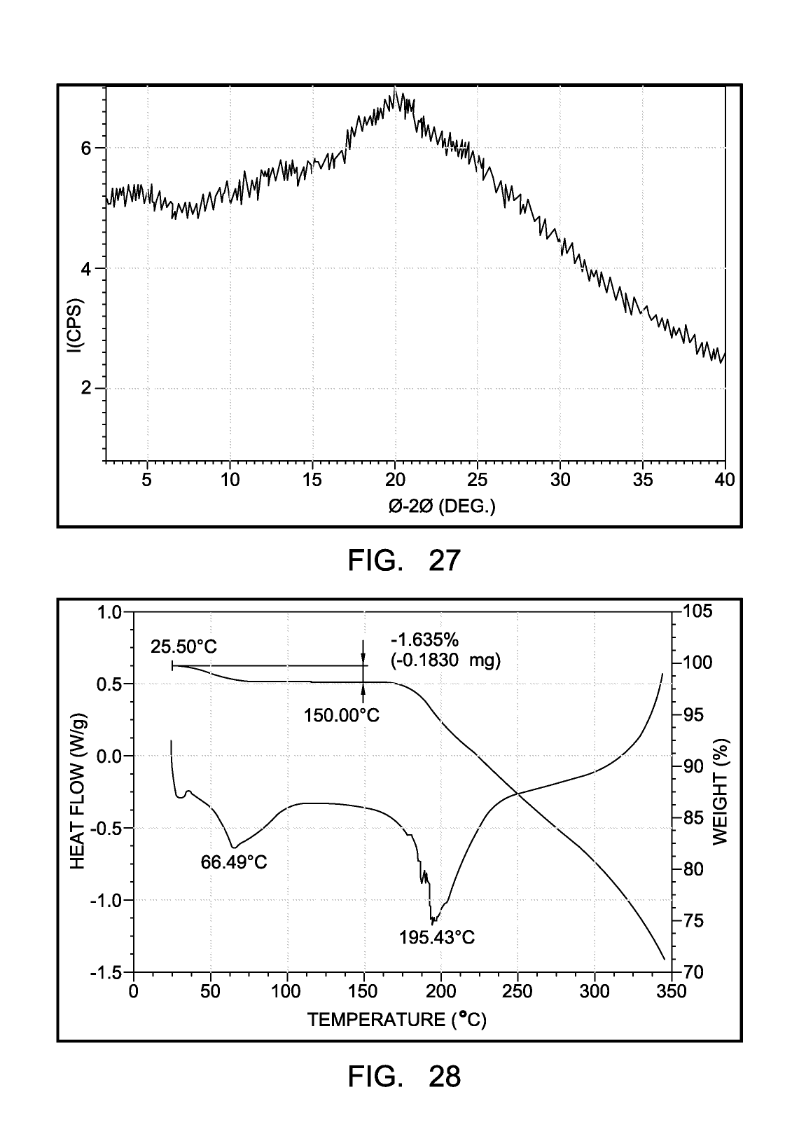

FIG. 27 illustrates the XRPD pattern of amorphous Form 2 (Sample No. 1888-41-03). The XRPD data show poor signal-to-noise ratio. The XRPD pattern tends to indicate that Form 2 is an amorphous form of Compound I.

Thermogravimetric and DSC data for amorphous Form 2 is summarized below in Table 1G and plotted in FIG. 28.

TABLE 1G
Thermal Data for Amorphous Form 2

| DSC Results¹ | TGA Results² |
|---|---|
| Endotherms at 66.5 and 195.4 | 1.6% at 150° C. |

¹Maximum temperature reported for transition
²Percent weight change from 35° C. to 200° C.

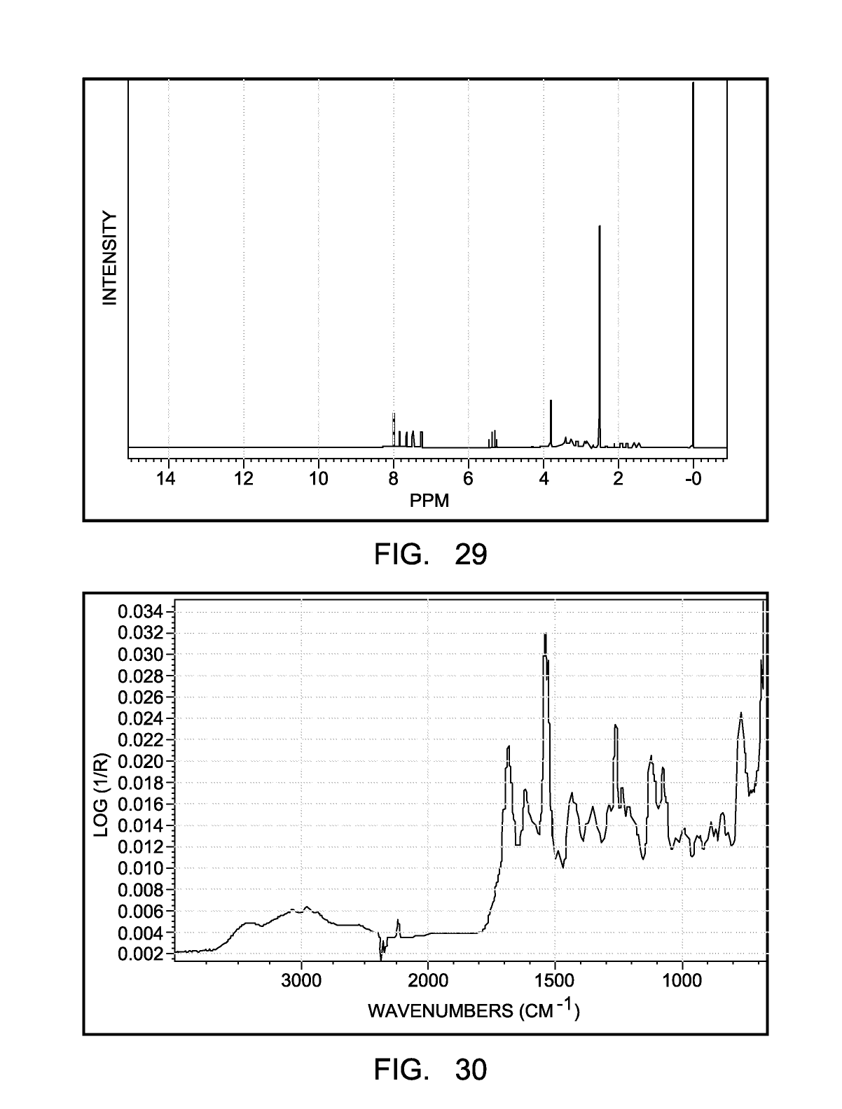

Referring to FIG. 29, the solution NMR spectrum of the amorphous material prepared by milling Form A (sample no. 1888-47-03) contains resonances that are attributable to Compound I, which indicates that the sample does not correspond to a decomposition product.

The IR spectrum for amorphous Form 2 (sample no. 1888-41-03) is plotted in FIG. 30. In one variation, IR peak positions that show no other peak within ±4 cm⁻¹ make up a unique set of peaks that can be used to identify the polymorph of Compound I.

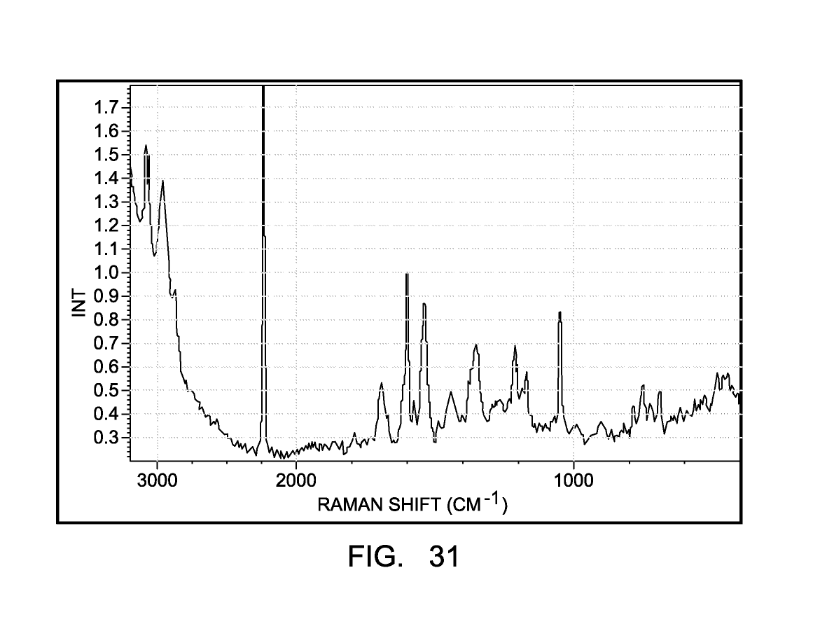

The Raman spectrum for amorphous Form 2 (sample no. 1888-41-03) is provided in FIG. 31. In one variation, Raman peak positions that show no other peak within ±4 cm⁻¹ make up a unique set of peaks that can be used to identify the polymorph of Compound I.

Data regarding the moisture sorption/desorption properties of amorphous Form 2 are summarized in Table 2F below.

TABLE 2F
Moisture Sorption/Desorption Data for Amorphous Form 2

| Elapsed Time | Weight | Weight | Sample Temperature | Sample |
|---|---|---|---|---|
| (min) | (mg) | Change (%) | (° C.) | RH (%) |
| 0.1 | 1.774 | 0.00 | 24.87 | 9.54 |
| 67.1 | 1.776 | 0.156 | 24.83 | 5.16 |
| 620.4 | 1.880 | 6.013 | 24.80 | 94.69 |
| 1346.1 | 1.781 | 0.433 | 24.84 | 5.10 |

4. Pharmaceutical Compositions Comprising Compound I Where at Least a Particular One of Form A, Form B, Form C, Form D, Form E, Form F, amorphous Form 1 or Amorphous Form 2 is Present

The polymorphs of the present invention may be used in various pharmaceutical compositions. Such pharmaceutical compositions may comprise Compound I present in the composition in a range of between 0.005% and 100% (weight/weight), with the balance of the pharmaceutical composition comprising additional substances such as those described herein. In particular variations, the pharmaceutical composition comprises at least 0.25%, 0.5%, 1%, 5%, 10%, 25%, 50%, 75%, 80%, 85%, 90%, 95%, 97%, or 99% of Compound I. A given one of the polymorphic forms of Compound I may comprise at least 1%, 5%, 10%, 25%, 50%, 75%, 80%, 85%, 90%, 95%, 97%, or 99% of Compound I (weight/weight) in the pharmaceutical composition.

In general, the pharmaceutical compositions of the present invention may be prepared in a gaseous, liquid, semi-liquid, gel, or solid form, and formulated in a manner suitable for the route of administration to be used where at least a portion of Compound I is present in the composition in a particular polymorph form.

Pharmaceutical compositions according to the present invention may be adapted for administration by any of a variety of routes. For example, pharmaceutical compositions according to the present invention can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery (for example, by catheter or stent), subcutaneously, intraadiposally, intraarticularly, or intrathecally, optionally in a slow release dosage form. In particular embodiments, the pharmaceutical compounds are administered orally, by inhalation or by injection subcutaneously, intramuscularly, intravenously or directly into the cerebrospinal fluid.

In addition to Compound I, the pharmaceutical composition may comprise one or more additional components that do not deleteriously affect the use of Compound I. For example, the pharmaceutical compositions may include, in addition to Compound I, conventional pharmaceutical excipients; diluents; lubricants; binders; wetting agents; disintegrating agents; glidants; sweetening agents; flavoring agents; emulsifying agents; solubilizing agents; pH buffering agents; perfuming agents; surface stabilizing agents; suspending agents; and other conventional, pharmaceutically inactive agents. In particular, the pharmaceutical compositions may comprise lactose, sucrose, dicalcium phosphate, carboxymethylcellulose, magnesium stearate, calcium stearate, talc, starch, natural gums (e.g., gum acaciagelatin, glucose, molasses, polyvinylpyrrolidine, celluloses and derivatives thereof), povidone, crospovidones acetate, sodium citrate, cyclodextrine derivatives, sorbitan monolaurate, triethanolamine sodium acetate, triethanolamine oleate, and other such agents. Methods of preparing such dosage forms are known in the art, and will be apparent to those skilled in this art; for example, see Remington's Pharmaceutical Sciences, 19^{th} Ed. (Easton, Pa.: Mack Publishing Company, 1995). The pharmaceutical composition to be administered should, in any event, contain a sufficient quantity of Compound I to reduce dipeptidyl peptidases activity in vivo sufficiently to provide the desired therapeutic effect.

Compositions, according to the present invention, may be administered, or coadministered with other active agents. These additional active agents may include, for example, one or more other pharmaceutically active agents. Coadministration in the context of this invention is intended to mean the administration of more than one therapeutic agent, one of which includes Compound I. Such coadministration may also be coextensive, that is, occurring during overlapping periods of time or may be sequential, that is, occurring during non-overlapping periods of time.

The following are particular examples of oral, intravenous and tablet formulations that may optionally be used with Compound I. It is noted that these compositions may be varied depending on the indication for which the composition is to be used.

Exemplary capsule formulations are as follows:

12.5 mg of Compound I (weight of free base form) per capsule

| | | |
|---|---|---|
| (1) 2-[[2-[(3R)-3-Amino-1-piperidinyl]-5-fluoro-6-oxo- | 18.22 | mg |
| 1(6H)-pyrimidinyl]methyl]-benzonitrile mono-L- | | |
| tartrate | | |
| (2) Lactose Monohydrate, NF | 48.91 | mg |
| (3) Microcrystalline Cellulose, NF | 45.62 | mg |
| (4) Croscarmellose Sodium, NF | 6.25 | mg |
| (5) Copovidone Ph Eur, JP | 5.00 | mg |
| (6) Magnesium Stearate, NF | 1.00 | mg |
| (7) White Opaque/White Opaque, Size 2 Capsules | 1 | capsule |
| TOTAL (per capsule) | 125.00 | mg |

25 mg of Compound I (weight of free base form) per capsule

| | | |
|---|---|---|
| (1) 2-[[2-[(3R)-3-Amino-1-piperidinyl]-5-fluoro-6-oxo- | 36.88 | mg |
| 1(6H)-pyrimidinyl]methyl]-benzonitrile mono-L- | | |
| tartrate | | |
| (2) Lactose Monohydrate, NF | 97.37 | mg |
| (3) Microcrystalline Cellulose, NF | 91.25 | mg |
| (4) Croscarmellose Sodium, NF | 12.50 | mg |
| (5) Copovidone Ph Eur, JP | 10.00 | mg |
| (6) Magnesium Stearate, NF | 2.00 | mg |
| (7) White Opaque/White Opaque, Size 2 Capsules | 1 | capsule |
| TOTAL (per capsule) | 250.00 | mg |

100 mg of Compound I (weight of free base form) per capsule

| | | |
|---|---|---|
| (1) 2-[[2-[(3R)-3-Amino-1-piperidinyl]-5-fluoro-6-oxo- | 147.42 | mg |
| 1(6H)-pyrimidinyl]methyl]-benzonitrile mono-L- | | |
| tartrate | | |
| (2) Lactose Monohydrate, NF | 25.80 | mg |
| (3) Microcrystalline Cellulose, NF | 18.56 | mg |
| (4) Croscarmellose Sodium, NF | 10.75 | mg |
| (5) Copovidone Ph Eur, JP | 10.75 | mg |
| (6) Magnesium Stearate, NF | 1.72 | mg |
| (7) White Opaque/White Opaque, Size 2 Capsules | 1 | capsule |
| TOTAL (per capsule) | 215.00 | mg |

200 mg of Compound I (weight of free base form) per capsule

| | | |
|---|---|---|
| (1) 2-[[2-[(3R)-3-Amino-1-piperidinyl]-5-fluoro-6-oxo- | 294.84 | mg |
| 1(6H)-pyrimidinyl]methyl]-benzonitrile mono-L- | | |
| tartrate | | |
| (2) Lactose Monohydrate, NF | 51.60 | mg |
| (3) Microcrystalline Cellulose, NF | 37.10 | mg |
| (4) Croscarmellose Sodium, NF | 21.50 | mg |
| (5) Copovidone Ph Eur, JP | 21.50 | mg |
| (6) Magnesium Stearate, NF | 3.46 | mg |
| (7) White Opaque/White Opaque, Size 2 Capsules | 1 | capsule |
| TOTAL (per capsule) | 430.00 | mg |

Exemplary intravenous and tablet formulations are as follows:

INTRAVENOUS FORMULATION

| | |
|---|---|
| Compound of the Present Invention | 0.1-10 mg |
| Dextrose Monohydrate | q.s. to make isotonic |
| Citric Acid Monohydrate | 1.05 mg |
| Sodium Hydroxide | 0.18 mg |
| Water for Injection | q.s. to 1.0 mL |

TABLET FORMULATION

| | |
|---|---|
| Compound of the Present Invention | 1% |
| Microcrystalline Cellulose | 73% |
| Stearic Acid | 25% |
| Colloidal Silica | 1% |

Provided in the examples are, by way of illustration but not limitation, more particular examples of formulations incorporating one or more of Form A, Form B, Form C, Form D, Form E, Form F, amorphous Form 1 or amorphous Form 2.

5. Indications for Use of Compound I

In one embodiment, Compound I and compositions, kits and articles of manufacture comprising Compound I are used to inhibit DPP-IV. Compound I and compositions, kits and articles of manufacture comprising Compound I are also used to treat a disease state for which DPP-IV possesses activity that contributes to the pathology and/or symptomology of the disease state.

Compound I may be administered to a subject wherein DPP-IV activity within the subject is altered, preferably reduced.

In another embodiment, a therapeutic method is provided that comprises administering Compound I. In another embodiment, a method of inhibiting cell proliferation is provided that comprises contacting a cell with an effective amount of Compound I. In another embodiment, a method of inhibiting cell proliferation in a patient is provided that comprises administering to the patient a therapeutically effective amount of Compound I.

In another embodiment, a method of treating a condition in a patient which is known to be mediated by DPP-IV, or which is known to be treated by DPP-IV inhibitors, comprising administering to the patient a therapeutically effective amount of Compound I. In another embodiment, a method is provided for using Compound I in order to manufacture a medicament for use in the treatment of disease state which is known to be mediated by DPP-IV, or which is known to be treated by DPP-IV inhibitors.

In another embodiment, a method is provided for treating a disease state for which DPP-IV possesses activity that contributes to the pathology and/or symptomology of the disease state, the method comprising: administering Compound I to a subject such that the free base form of Compound I is present in the subject in a therapeutically effective amount for the disease state.

In another embodiment, a method is provided for treating a cell proliferative disease state comprising administering Compound I so that cells are treated with the free base form of Compound I in combination with an anti-proliferative agent, wherein the cells are treated with the free base form of Compound I, at the same time, and/or after the cells are treated with the anti-proliferative agent, referred to herein as combination therapy. It is noted that treatment of one agent before another is referred to herein as sequential therapy, even if the agents are also administered together. It is noted that combination therapy is intended to cover when agents are administered before or after each other (sequential therapy) as well as when the agents are administered at the same time.

Examples of diseases that may be treated by administration of Compound I and compositions according to the present invention include, but are not limited to conditions mediated by DPP-IV, in particular diabetes, more particular type 2 diabetes mellitus, diabetic dislipidemia, conditions of impaired glucose tolerance (IGT), conditions of impaired fasting plasma glucose (IFG), metabolic acidosis, ketosis, appetite regulation, obesity, immunosuppressants or cytokine release regulation, autoimmune diseases such as inflammatory bowel disease, multiple sclerosis and rheumatoid arthritis, AIDS, cancers (prevention of metastases, for example, breast and prostrate tumors to the lungs), dermatological diseases such as psoriasis and lichen planus, treatment of female infertility, osteoporosis, male contraception and neurological disorders.

6. Kits and Articles of Manufacture Comprising Compound I Polymorphs

The present invention is also directed to kits and other articles of manufacture for treating diseases associated with dipeptidyl peptidases. It is noted that diseases are intended to cover all conditions for which the dipeptidyl peptidases possesses activity that contributes to the pathology and/or symptomology of the condition.

In one embodiment, a kit is provided that comprises a pharmaceutical composition comprising Compound I where greater than 1%, 5%, 10%, 25%, 50%, 75%, 80%, 85%, 90%, 95%, 97% or 99% of Compound I (by weight) is present in the composition as a particular one of Form A, Form B, Form C, Form D, Form E, Form F, amorphous Form 1 or amorphous Form 2; and instructions for use of the kit. Optionally, the composition comprises at least 0.25%, 0.5%, 1%, 5%, 10%, 25%, 50%, 75%, 80%, 85%, 90%, 95%, 97%, or 99% of Compound I. The instructions may indicate the disease state for which the composition is to be administered, storage information, dosing information and/or instructions regarding how to administer the composition. The kit may also comprise packaging materials. The packaging material may comprise a container for housing the composition. The kit may also optionally comprise additional components, such as syringes for administration of the composition. The kit may comprise the composition in single or multiple dose forms.

In another embodiment, an article of manufacture is provided that comprises a pharmaceutical composition comprising Compound I where greater than 1%, 5%, 10%, 25%, 50%, 75%, 80%, 85%, 90%, 95%, 97% or 99% of Compound I (by weight) is present in the composition as a particular one of Form A, Form B, Form C, Form D, Form E, Form F, amorphous Form 1 or amorphous Form 2; and packaging materials. Optionally, the composition comprises at least 0.25%, 0.5%, 1%, 5%, 10%, 25%, 50%, 75%, 80%, 85%, 90%, 95%, 97%, or 99% of Compound I. The packaging material may comprise a container for housing the composition. The container may optionally comprise a label indicating the disease state for which the composition is to be administered, storage information, dosing information and/or instructions regarding how to administer the composition. The article of manufacture may also optionally comprise additional components, such as syringes for administration of the composition. The article of manufacture may comprise the composition in single or multiple dose forms.

It is noted that the packaging material used in kits and articles of manufacture according to the present invention may form a plurality of divided containers such as a divided bottle or a divided foil packet. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container that is employed will depend on the exact dosage form involved, for example a conventional cardboard box would not generally be used to hold a liquid suspension. It is feasible that more than one container can be used together in a single package to market a single dosage form. For example, tablets may be contained in a bottle that is in turn contained within a box. Typically the kit includes directions for the administration of the separate components. The kit form is particularly advantageous when the separate components are preferably administered in different dosage forms (e.g., oral, topical, transdermal and parenteral), are administered at different dosage intervals, or when titration of the individual components of the combination is desired by the prescribing physician.

One particular example of a kit according to the present invention is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of individual tablets or capsules to be packed or may have the size and shape to accommodate multiple tablets and/or capsules to be packed. Next, the tablets or capsules are placed in the recesses accordingly and the sheet of relatively stiff material is sealed against the plastic foil at the face of the foil which is opposite from the direction in which the recesses were formed. As a result, the tablets or capsules are individually sealed or collectively sealed, as desired, in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

Another specific embodiment of a kit is a dispenser designed to dispense the daily doses one at a time in the order of their intended use. Preferably, the dispenser is equipped with a memory-aid, so as to further facilitate compliance with the regimen. An example of such a memory-aid is a mechanical counter that indicates the number of daily doses that has been dispensed. Another example of such a memory-aid is a battery-powered micro-chip memory coupled with a liquid crystal readout, or audible reminder signal which, for example, reads out the date that the last daily dose has been taken and/or reminds one when the next dose is to be taken.

7. Dosage Forms

Compositions according to the present invention are optionally provided for administration to humans and animals in unit dosage forms or multiple dosage forms, such as tablets, capsules, pills, powders, dry powders for inhalers, granules, sterile parenteral solutions or suspensions, oral solutions or suspensions, and oil-water emulsions containing suitable quantities Compound I. Unit-dose forms, as used herein, refers to physically discrete units suitable for human and animal subjects and packaged individually as is known in the art. Each unit-dose contains a predetermined quantity of Compound I sufficient to produce the desired therapeutic effect, in association with a pharmaceutical carrier, vehicle or diluent. Examples of unit-dose forms include ampoules and syringes, and individually packaged tablets or capsules. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules, or bottles of pints or gallons. Hence, multiple dose form may be viewed as a multiple of unit-doses that are not segregated in packaging.

In general, the total amount of Compound I in a pharmaceutical composition according to the present invention should be sufficient to a desired therapeutic effect. This amount may be delivered as a single per day dosage, multiple dosages per day to be administered at intervals of time, or as a continuous release dosage form. Dosage forms or compositions may optionally comprise Compound I in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of Compound I, optionally 0.1-95%, and optionally 1-95%. Compositions according to the present invention are optionally provided for administration to humans and animals in unit dosage forms, such as tablets, capsules, pills, powders, dry powders for inhalers, granules, sterile parenteral solutions or suspensions, and oral solutions or suspensions, and oil-water emulsions containing suitable quantities of the compounds, particularly the pharmaceutically acceptable salts, preferably the sodium salts, thereof. The pharmaceutically therapeutically active compounds and derivatives thereof are typically formulated and administered in unit-dosage forms or multiple-dosage forms. Unit-dose forms, as used herein, refers to physically discrete units suitable for human and animal subjects and packaged individually as is known in the art. Each unit-dose contains a predetermined quantity of the therapeutically active compound sufficient to produce the desired therapeutic effect, in association with the required pharmaceutical carrier, vehicle or diluent. Examples of unit-dose forms include ampoules and syringes individually packaged tablet or capsule. Unit-dose forms may be administered in fractions or multiples thereof. A multiple-dose form is a plurality of identical unit-dosage forms packaged in a single container to be administered in segregated unit-dose form. Examples of multiple-dose forms include vials, bottles of tablets or capsules or bottles of pint or gallons. Hence, multiple dose form is a multiple of unit-doses that are not segregated in packaging. Dosage forms or compositions may optionally comprise Compound I in the range of 0.005% to 100% (weight/weight) with the balance comprising additional substances such as those described herein. For oral administration, a pharmaceutically acceptable composition may optionally comprise any one or more commonly employed excipients, such as, for example pharmaceutical grades of mannitol, lactose, starch, magnesium stearate, talcum, cellulose derivatives, sodium crosscarmellose, glucose, sucrose, magnesium carbonate, sodium saccharin, talcum. Such compositions include solutions, suspensions, tablets, capsules, powders, dry powders for inhalers and sustained release formulations, such as, but not limited to, implants and microencapsulated delivery systems, and biodegradable, biocompatible polymers, such as collagen, ethylene vinyl acetate, polyanhydrides, polyglycolic acid, polyorthoesters, polylactic acid and others. Methods for preparing these formulations are known to those skilled in the art. The compositions may optionally contain 0.01%-100% (weight/weight) of Compound I, optionally 0.1-95%, and optionally 1-95%.

In one embodiment, the pharmaceutical composition is a pill or capsule adapted for oral administration. In another embodiment, the pharmaceutical composition is in an oral dosage form selected from the group consisting of pills, tablets, capsules, emulsions, suspensions, microsuspensions, wafers, sprinkles, chewing gum, powders, lyophilized powders, granules, and troches. In still another embodiment, the pharmaceutical composition is in a parenteral dosage form selected from the group consisting of suspensions, microsuspensions, emulsions, solid forms suitable for suspension or emulsification prior to injection, and implantable devices. In yet another embodiment, the pharmaceutical composition is adapted for topical or transdermal administration. In a further embodiment, the pharmaceutical composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches. In still a further embodiment, the pharmaceutical composition is in a pulmonary dosage form selected from the group consisting of powders, aerosols, suspensions, microsuspensions, and emulsions.

A. Formulations for Oral Administration

Oral pharmaceutical dosage forms may be as a solid, gel or liquid where Compound I is retained in one of the polymorphic forms. Examples of solid dosage forms include, but are not limited to tablets, capsules, granules, and bulk powders. More specific examples of oral tablets include compressed, chewable lozenges and tablets that may be enteric-coated, sugar-coated or film-coated. Examples of capsules include hard or soft gelatin capsules. Granules and powders may be provided in non-effervescent or effervescent forms. Each may be combined with other ingredients known to those skilled in the art.

In certain embodiments, Compound I is provided as solid dosage forms, preferably capsules or tablets. The tablets, pills, capsules, troches and the like may optionally contain one or more of the following ingredients, or compounds of a similar nature: a binder; a diluent; a disintegrating agent; a lubricant; a glidant; a sweetening agent; and a flavoring agent.

Examples of binders that may be used include, but are not limited to, microcrystalline cellulose, gum tragacanth, glucose solution, acacia mucilage, gelatin solution, sucrose and starch paste.

Examples of lubricants that may be used include, but are not limited to, talc, starch, magnesium or calcium stearate, lycopodium and stearic acid.

Examples of diluents that may be used include, but are not limited to, lactose, sucrose, starch, kaolin, salt, mannitol and dicalcium phosphate.

Examples of glidants that may be used include, but are not limited to, colloidal silicon dioxide.

Examples of disintegrating agents that may be used include, but are not limited to, crosscarmellose sodium, sodium starch glycolate, alginic acid, corn starch, potato starch, bentonite, methylcellulose, agar and carboxymethylcellulose.

Examples of coloring agents that may be used include, but are not limited to, any of the approved certified water soluble FD and C dyes, mixtures thereof, and water insoluble FD and C dyes suspended on alumina hydrate.

Examples of sweetening agents that may be used include, but are not limited to, sucrose, lactose, mannitol and artificial sweetening agents such as sodium cyclamate and saccharin, and any number of spray-dried flavors.

Examples of flavoring agents that may be used include, but are not limited to, natural flavors extracted from plants such as fruits and synthetic blends of compounds that produce a pleasant sensation, such as, but not limited to peppermint and methyl salicylate.

Examples of wetting agents that may be used include, but are not limited to, propylene glycol monostearate, sorbitan monooleate, diethylene glycol monolaurate and polyoxyethylene lauryl ether.

Examples of anti-emetic coatings that may be used include, but are not limited to, fatty acids, fats, waxes, shellac, ammoniated shellac and cellulose acetate phthalates.

Examples of film coatings that may be used include, but are not limited to, hydroxyethylcellulose, sodium carboxymethylcellulose, polyethylene glycol 4000 and cellulose acetate phthalate.

If oral administration is desired, Compound I may optionally be provided in a composition that protects it from the acidic environment of the stomach. For example, the composition can be formulated in an enteric coating that maintains its integrity in the stomach and releases the active compound in the intestine. The composition may also be formulated in combination with an antacid or other such ingredient.

When the dosage unit form is a capsule, it may optionally additionally comprise a liquid carrier such as a fatty oil. In addition, dosage unit forms may optionally additionally comprise various other materials that modify the physical form of the dosage unit, for example, coatings of sugar and other enteric agents.

Compound I may also be administered as a component of an elixir, suspension, syrup, wafer, sprinkle, chewing gum or the like. A syrup may optionally comprise, in addition to the active compounds, sucrose as a sweetening agent and certain preservatives, dyes and colorings and flavors.

It is understood that the precise dosage and duration of treatment will be a function of the location of where the composition is parenterally administered, the carrier and other variables that may be determined empirically using known testing protocols or by extrapolation from in vivo or in vitro test data. It is to be noted that concentrations and dosage values may also ultimately depend on, among other criteria known to those of skill in the art, the age, weight and condition of the patient or animal, as is known in the art. It is to be further understood that for any particular subject, specific dosage regimens may need to be adjusted over time according to the individual need and the professional judgment of the person administering or supervising the administration of the formulations. Hence, the concentration ranges set forth herein are intended to be exemplary and are not intended to limit the scope or practice of the claimed formulations.

### EXAMPLES

### Example 1

### Preparation of Compound I

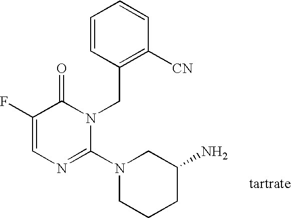

### Example 1a

### 2-Chloro-5-fluoro-3H-pyrimidin-4-one

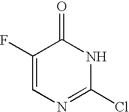

The title compound was prepared in 56% yield from 2,4-dichloro-5-fluoro-pyrimidine as described in U.S. patent application Ser. No. 10/918,317. Specifically, 5-Fluoro-2,4-dichloro-pyrimidine was stirred in THF (10 mL) with 1N NaOH at r.t. for 3 h. The solution was made slightly acidic with 1N HCl and was extracted with CHCl₃. Organics were dried (MgSO₄) and concentrated in vacuo. Precipitation from 20% CHCl₃/hexanes and collection by filtration gave the title compound. ¹H NMR (400 MHz, DMSO-d₆): δ 13.98 (br s, 1H), 8.14 (d, 1H, J=3.2 Hz).

The title compound of Example 1a was also prepared as follows. Dimethylaniline (195 mL, 1.54 mol) was added to a slurry of 5-fluorouracil (99.73 g, 0.77 mol) in phosphorus oxychloride (215 mL, 2.31 mol) at 95° C. under a nitrogen atmosphere. The reaction mixture was stirred at this temperature for 3.5 h, cooled to room temperature and then slowly added to a stirred mixture of ice (200 g) and 6 M HCl (200 mL). The resulting slurry was extracted with dichloromethane (2×400 mL) and the combined organic extracts were washed with DI water (4×275 mL), dried over MgSO₄ and concentrated under reduced pressure to afford 111.77 g (87%, 98.1% AUC by HPLC) of 5-fluoro-2,4-dichloro-pyrimidine as an amber oil. A molar solution of sodium hydroxide (1.34 L) was slowly added to a solution of the 5-fluoro-2,4-dichloro-pyrimidine (111.77 g, 0.67 mol) in THF (377 mL) at 0° C. After the reaction mixture was stirred for about 30 min at room temperature, the pH was adjusted to 6 by a slow addition of 1.0 M HCl. The aqueous solution was extracted with ethyl acetate (440 mL) to remove impurities following which the pH was adjusted to 1 with 1.0 M HCl. The acidic aqueous solution was extracted with ethyl acetate (4×555 mL) and the combined organic extracts were washed with brine (111 mL), dried over MgSO₄ and concentrated under reduced pressure to produce 88.35 g (89%, 99.2% AUC by HPLC) of 2-chloro-5-fluoro-3H-pyrimidin-4-one as an off-white powder.

### Example 1b

### 2-(2-Chloro-5-fluoro-6-oxo-6H-pyrimidin-1-ylmethyl)-benzonitrile

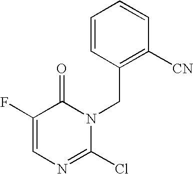

The title compound was prepared in 44% yield from 2-chloro-5-fluoro-3H-pyrimidin-4-one as described in U.S. patent application Ser. No. 10/918,317. Specifically, 5-Fluoro-2-chloro-3H-pyrimidin-4-one was stirred in DME/DMF under nitrogen at 0° C. Sodium hydride (95%) was added in portions. After 10 min, lithium bromide was added and the reaction stirred at r.t. α-Bromo-o-tolunitrile was added, and the reaction stirred at 65° C. for 8 h. The solution was diluted with EtOAc, washed with brine, dried (MgSO₄) and concentrated in vacuo. Purification by silica gel chromatography (1:1:1 EtOAc/hexanes/CHCl₃) gave the title compound. Also obtained from the reaction were impure fractions of the less polar O-alkylated isomer, and the more polar N3-alkylated isomer. ¹H NMR (400 MHz, CDCl₃): δ 7.81 (s, 1H), 7.74 (dd, 1H, J=7.6, 1.2 Hz), 7.59 (td, 1H, J=7.6, 1.2 Hz), 7.45 (t, 1H, J=7.6 Hz), 7.15 (d, 1H, J=7.6 Hz), 5.67 (s, 2H). MS (ES) [m+H] calc'd for C₁₂H₇N₃OFCl, 264, 266; found 264, 266.

The title compound of Example 1b was also prepared from 5-fluoro-2-chloro-3H-pyrimidin-4-one as follows. To a solution of 5-fluoro-2-chloro-3H-pyrimidin-4-one (100.00 g, 0.67 mol) in 1:1 DMF/DME (440 mL) under a nitrogen atmosphere was added sodium iodide (10 g, 67 mmol) and the resulting slurry cooled to 0° C. While maintaining the internal temperature to ≦12° C., 1,1,3,3-tetramethylguanidine (94 mL, 0.74 mol) was added dropwise over 45 minutes to form a homogeneous solution. The ice bath was removed and a solution of 2-(bromomethyl)benzonitrile (145 g, 0.74 mol) in 1:1 DMF/DME (600 mL) was added all at once and the reaction mixture heated to 70° C. for 18 h. In-process assay (by HPLC analysis) showed complete consumption of the starting material. The reaction mixture was cooled to 30° C. and then MTBE (1.14 L) and DI water (2.29 L) were added to form a slurry which was stirred at ambient temperature for 1 h. The crude product was collected by filtration and washed with MTBE (1.14 L). The phases of the filtrate were separated and the organic layer was combined with the filter-cake and concentrated to a thick slurry which was subsequently reslurried with DME (1.00 L). Insoluble impurities were removed by vacuum filtration and washed with DME (500 mL). The filtrate was concentrated to a wet residue which was reslurried with 2-propanol (365 mL) and the solid product collected by vacuum filtration. After removal of residual IPA, the product was reslurried in ethyl acetate (6 vol) at 70° C. and heptane (6 vol) was added slowly while maintaining the internal temperature at 68-70° C. At the end of the addition of heptane, insoluble materials were still visible. The mixture was stirred for an additional 10 min after which insoluble impurities were removed by hot vacuum filtration. The filtrate was slowly cooled to room temperature and then cooled further to about 0° C. by means of a salt-ice bath. The crystallized product was collected by vacuum filtration and then reslurried in ethanol (6 vol) at 50° C., cooled to 40° C., and the product collected by vacuum filtration to give 80.62 g (45%, 95.6% AUC by HPLC) of 2-(2-chloro-5-fluoro-6-oxo-6H-pyrimidin-1-ylmethyl)-benzonitrile as an off-white solid.

### Example 1c

### 2- [2-(3-(R)-Amino-piperidin-1-yl)-5-fluoro-6-oxo-6H-pyrimidin-1-ylmethyl]-benzonitrile

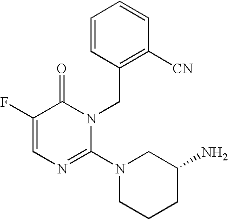

The title compound of Example 1c was prepared in 68% yield from 2-(2-chloro-5-fluoro-6-oxo-6H-pyrimidin-1-ylmethyl)-benzonitrile and was isolated as the TFA salt as described in U.S. Patent Publication No. US 2005/0070535. Specifically, 2-(5-fluoro-2-chloro-6-oxo-6H-pyrimidin-1-ylmethyl)-benzonitrile, (R)-3-amino-piperidine, dihydrochloride and sodium bicarbonate were stirred in ethanol at 60° C. for 90 min. The reaction was diluted with EtOAc, washed with water and brine, dried (MgSO₄), and concentrated in vacuo. Purification by silica gel chromatography (5% MeOH/CHCl₃) gave the title compound. This was converted to the solid TFA salt by subjection to TFA in CH₂Cl₂ and concentration in vacuo. ¹H NMR (400 MHz, MeOD-d₄): δ 7.99 (d, 1H, J=0.8 Hz), 7.85 (d, 1H, J=7.2 Hz), 7.64 (t, 1H, J=7.6 Hz), 7.47 (t, 1H, J=7.6 Hz), 7.20 (d, 1H, J=7.6 Hz), 5.33 (s, 2H), 3.49-3.58 (m, 1H), 3.10-3.19 (m, 1H), 2.68-2.76 (m, 2H), 2.48-2.58 (m, 1H), 1.60-1.80 (m, 2H), 1.41-1.51 (m, 1H), 1.10-1.19 (m, 1H). MS (ES) [m+H] calc'd for C₁₇H₁₈N₅OF, 328; found 328.

The title compound of Example 1c was also prepared from 2-(2-chloro-5-fluoro-6-oxo-6H-pyrimidin-1-ylmethyl)-benzonitrile as follows. A mixture of 2-(2-chloro-5-fluoro-6-oxo-6H-pyrimidin-1-ylmethyl)-benzonitrile (80.62 g, 0.31 mol), (R)-3-aminopiperidine dihydrochloride (58.00 g, 0.34 mol) and potassium carbonate (186 g, 1.35 mol) in 10% water in IPA (807 mL) was heated at 45° C. for 1 h. After cooling to room temperature, isopropyl acetate (807 mL) and 2 M HCl (807 mL) were added. Following phase separation, the organic layer was extracted with 2.0 M HCl (2×807 mL). The aqueous extracts were combined, washed with isopropyl acetate (807 mL), cooled to 10° C. and the pH adjusted to 13 with caustic soda. The alkaline slurry was extracted with isopropyl acetate (2×807 mL), and the combined organic extracts concentrated to afford 93.50 g (93%, 98.4% AUC by HPLC) of the title compound of Example 1c as a viscous oil (93.49 g, 93%).

### Example 1d

### Tartrate salt of 2-[2-(3-(R)-Amino-piperidin-1-yl)-5-fluoro-6-oxo-6H-pyrimidin-1-ylmethyl]-benzonitrile

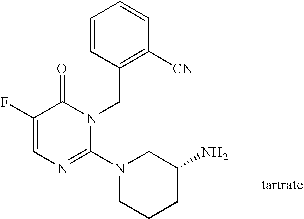

The title compound (Compound I) was prepared by adding a solution of L-tartaric acid in 5% water in IPA (3.00 L) to a solution of Example 1c (93.00 g, 284 mmol) in methanol (982 mL) at 65° C. The mixture was stirred for 20 min and then cooled to room temperature. The resulting precipitate was collected by vacuum filtration, washed with 5% water in IPA (2×560 mL), and dried in a vacuum oven at 75° C. to give 112.78 g (77%, 100% AUC by HPLC) of the salt as a white solid.

### Example 2

### Characterization of Solubility of Compound I in Different Solvents

The following experiments were performed in order to determine the solubility of Compound I in different solvents and solvent systems. This information was later used to identify potential crystallization conditions for Compound I.

### Materials and Reagents

Unless otherwise stated Compound I form A (lot no 2449-B-RO-01-48-10) and amorphous material prepared from this sample were used as the starting materials for all crystallization experiments. Solvents and other reagents were of ACS or HPLC grade and were used as received.

### Solubility Estimates

A weighed sample (typically about 20 mg) of Compound I form A was treated with aliquots of the test solvent at ambient temperature. The aliquot volumes were typically 250 μL. Periodically, the mixture was sonicated between additions to facilitate dissolution. Complete dissolution of the test material was determined by visual inspection. Solubilities were estimated from these experiments based on the total solvent used to provide complete dissolution. If dissolution did not occur after the last aliquot of solvent was added, the solubility is expressed as “less than” (<). If dissolution occurred after the last aliquot was added then solubility is expressed as “greater than or equal to” (≧). The solubility values for Compound I (Table 4) have been rounded to the nearest whole number. The true equilibrium solubilities for the compound may be different than the values provided here because of the use of solvent aliquots that were too large or due to a slow rate of dissolution.

TABLE 4
Solubility Estimates for Compound I Form A

| | | Solubility |
|---|---|---|
| Solvent | Acronym | (mg/mL)^{a} |
| acetone | — | <4 |
| acetonitrile | ACN | <3 |
| chloroform | — | <4 |
| dichloromethane | DCM | <3 |
| diisopropyl ether | DIPE | <3 |
| dimethylformamide | DMF | ≧81 |
| 1,4-dioxane | — | <3 |
| ethanol | EtOH | <3 |
| ethyl acetate | EtOAc | <3 |
| hexafluoroisopropanol | HFIPA | ≧80 |
| hexanes | — | <3 |
| isopropanol | IPA | <3 |
| isopropyl acetate | iPrOAc | <3 |
| methanol | MeOH | ≧16 |
| methyl ethyl ketone | MEK | <3 |
| nitromethane | — | <3 |
| tetrahydrofuran | THF | <3 |
| toluene | — | <3 |
| trifluoroethanol | TFE | ≧81 |
| water | — | ≧41 |

^{a}Data reported to the nearest mg/mL

Form A polymorph was found to be soluble in water (≧41 mg/mL), methanol (≧16 mg/mL), dimethylformide (≧81 mg/mL), 1,1,1,3,3,3-hexafluoroisopropanol (≧80 mg/mL) and 2,2,2-trifluroethanol (≧81 mg/mL). Thermal analysis indicates that this solid phase is thermally stable up to about 200° C. DSC analysis and melting point determinations determined that form A melts at around 172° C. Moisture sorption/desorption analysis of form A demonstrates that this polymorph is a variable hydrate.

TABLE 5
Compound I Polymorph Screen - Solution-Based Experiments

| | | XRPD | |
|---|---|---|---|
| Solvent^{a} | Conditions^{b} | Pattern | Sample No. |
| acetone | Slurry, ambient, 14 d | Form A | 1888-68-04 |
| acetone:water (2:3) | FE, ambient | Form A | 1888-46-01 |
| ACN | Slurry, ambient, 14 d | Form A | 1888-68-07 |
| | FE, ambient | Form B | 1888-46-02 |
| | FE, ambient | Form E | 1888-84-01 |
| | FE, ambient | Form B | 1982-09-01 |
| | FE, ambient | Form B | 1982-20-01 |
| chloroform | Slurry, ambient, 14 d | Form A | 1888-68-05 |
| DCM | Slurry, ambient, 14 d | Form A | 1888-68-08 |
| diisopropyl ether | Slurry, ambient, 13 d | Form A | 1888-69-01 |
| | Slurry, ambient, 13 d | Form A | 1888-69-03 |
| | Vapor stress, amorphous | amorphous | 1888-71-06 |
| | sample no 1888-47-01, | | |
| | ambient, 21 d | | |
| | FE, ambient | Form C | 1888-46-03 |
| | FE, ambient | Form B | 1888-84-02 |
| | FE, ambient | Form E | 1982-09-02 |
| | SE, ambient | gum | 1888-48-04 |
| | Set aside, ambient | no solids | 1888-55-01 |
| | Slurry, ambient, 14 d | Form A | 1888-68-03 |
| | Vapor stress, amorphous | amorphous | 1888-71-04 |
| | sample no 1888-47-01, | | |
| | ambient, 21 d | | |
| | Slurry, ambient, 14 d | Form A | 1888-68-01 |
| | Vapor stress, amorphous | amorphous | 1888-71-03 |
| | sample no 1888-47-01, | | |
| | ambient, 21 d | | |
| | Crystallization via | amorphous | 1888-93-05 |
| | amorphous material | | |
| ethanol:water (1:1) | FE, ambient | Form C | 1888-46-06 |
| hexanes | Slurry, ambient, 14 d | Form A | 1888-68-02 |
| HFIPA | SE, ambient | Form A | 1888-48-05 |
| HFIPA:hexanes (1:1) | SE, ambient | gum | 1888-65-03 |
| HFIPA:IPA (1:1) | SE, ambient | gum | 1888-65-04 |
| HFIPA:DCM (1:1) | SE, ambient | gum | 1888-65-02 |
| HFIPA:nitromethane | SE, ambient | gum | 1888-65-01 |
| | Slurry, ambient, 14 d | Form A | 1888-68-06 |
| | Crystallization via | amorphous | 1888-93-03 |
| | amorphous material | | |
| | FE, ambient | Form C | 1888-46-04 |
| | FE, ambient | Form C | 1982-11-01 |
| | FE, ambient | Form B | 1982-20-02 |
| iPrOAc | Slurry, ambient, 13 d | Form A | 1888-69-05 |
| | SE, ambient | Form A | 1888-48-03 |
| | FEP, ambient | Form A | 1888-49-02 |
| | SEP, ambient | Form A | 1888-49-04 |
| | Crystallization via | amorphous | 1888-93-04 |
| | amorphous material | | |
| | SC plus ultrasound | Form A | 1888-80-01 |
| | CC plus ultrasound | Form A | 1888-80-02 |
| | slurry ambient, 21 d | Form A | 1982-10-01 |
| | Slurry, ambient, 22 d | Form A | 1982-26-03 |
| | Vapor stress, amorphous | Form D | 1888-71-02 |
| | sample no 1888-47-01, | | |
| | ambient, 21 d | | |
| methanol/acetone | S/AS | Form A | 1888-51-02 |
| methanol:ACN (1:1) | FE, ambient | Form A | 1888-64-01 |
| methanol:dioxane (1:1) | FE, ambient | Form A | 1888-64-04 |
| methanol:EtOAc (1:1) | FE, ambient | Form A | 1888-64-03 |
| methanol:ethanol (1:1) | FE, ambient | Form A | 1888-64-02 |
| methanol:toluene | S/AS | Form A | 1888-51-03 |
| MEK | Slurry, ambient, 14 d | Form A | 1888-69-07 |
| nitromethane | Slurry, ambient, 13 d | Form A | 1888-69-06 |
| TFE | SE, ambient | Form A | 1888-48-02 |
| | Slurry, ambient, 13 d | Form A | 1888-69-02 |
| | Vapor stress, amorphous | amorphous | 1888-71-05 |
| | sample no 1888-47-01, | | |
| | ambient, 21 d | | |
| | FE, ambient | Form E | 1982-11-02 |
| | FE, ambient | Form B | 1982-20-03 |
| THF:water (2:1) | FE, ambient | Form B | 1888-46-05 |
| toluene | Slurry, ambient, 13 d | Form A | 1888-69-04 |
| | Freeze dried material | amorphous | 1888-47-01 |
| | SE, ambient | Form A | 1888-48-01 |
| | FEP, ambient | Form A | 1888-49-01 |
| | SEP, ambient | Form A + B | 1888-49-03 |
| | SC | no solid | 1888-50-01 |
| | SC | Form A | 1888-93-01 |
| | MC | Form A | 1888-93-02 |
| | CC | Form D | 1888-50-03 |
| | Vapor stress, amorphous | Form A | 1888-71-01 |
| | sample no 1888-47-01, | | |
| | ambient, 21 d | | |
| | CC | Form B | 1888-85-01 |
| 16% water in ACN | Slurry 50° C. 21 d | Form A | 1982-22-02 |
| | FE, ambient | Form E | 1982-05-01 |
| | CC | Form A | 1982-05-02 |
| | FE, 60° C. | gum | 1982-05-03 |
| | Slurry, temp recycle, 20-75° C., | decomposed^{c} | 1982-13-02 |
| | 7 d | | |
| 1.5% water in DCM | Slurry, ambient, 21 d | Form A | 1982-22-06 |
| | Slurry, 50° C., 21 d | Form A | 1982-22-03 |
| | Slurry, temp recycle, 20-75° C., | decomposed^{c} | 1982-13-03 |
| | 7 d | | |
| | Slurry, 50° C., 7 d | Form A | 1982-22-04 |
| | Slurry, temp recycle, 20-75° C., | decomposed^{c} | 1982-13-04 |
| | 7 d | | |
| | Slurry, 50° C., 7 d | Form A | 1982-22-01 |
| | Slurry, temp recycle, 20-75° C., | decomposed^{c} | 1982-13-01 |
| | 7 d | | |
| | Slurry, 50° C., 7 d | Form A | 1982-22-06 |
| | | (weak signal) | |
| | Slurry, 50° C., 21 d | Form A | 1982-22-05 |
| | Slurry, temp recycle, 20-75° C., | decomposed^{c} | 1982-13-05 |
| | 7 d | | |

^{a}Solvent mixtures correspond to a volume:volume ratio
^{b}CC = Crash Cool; FE = Fast Evaporation; Sat soln = saturated solution at ambient; SC = Slow Cool; SE = Slow Evaporation, S/AS = Solvent/Antisolvent; d = days

### Sample Preparation

General Methods

Fast Evaporation (FE): A solution of Compound I was prepared in a given solvent and filtered through a 0.2-μm nylon filter. The filtered solution was allowed to evaporate to dryness in a fume hood at ambient temperature in an open vial.

Slow Evaporation (SE): A solution of Compound I was prepared in a given solvent and filtered through a 0.2-μm nylon filter. The filtered solution was allowed to evaporate to dryness in a fume hood at ambient temperature in a vial that was either capped loosely or covered with a piece of aluminum foil containing pinholes.

Fast Evaporation Precipitation (FEP): A solution of Compound I was prepared in a given solvent and filtered through a 0.2-μm nylon filter. The filtered solution was allowed to evaporate in a fume hood at ambient temperature in an open vial. Solid material was isolated by filtration before complete evaporation of the solvent.

Slow Evaporation Precipitation (SEP): A solution of Compound I was prepared in a given solvent and filtered through a 0.2-μm nylon filter. The filtered solution was allowed to evaporate in a fume hood at ambient temperature in a vial that was either capped loosely or covered with a piece of aluminum foil containing pinholes. Solid material was isolated by filtration before complete evaporation of the solvent.

Slow Cool (SC): A solution of Compound I was prepared in a given solvent at an elevated temperature. The solution was filtered through a 0.2-μm filter into an open vial while still warm. The vial was sealed and allowed to cool slowly. Solids that formed were isolated by vacuum filtration and air-dried prior to analysis.

Crash Cool (CC): Solutions of Compound I were prepared in various solvents and filtered through a 0.2-μm filter. Adding the filtered solution to a vial and immediately placing the sample into a dry ice/acetone bath for several minutes induced solid formation. The resulting solids were isolated by vacuum filtration and air-dried in the fume hood prior to analysis.

Solvent/Anti-Solvent Crash (S/AS crash): Solutions of Compound I were prepared in various solvents and filtered through a 0.2-μm filter. Adding the filtered solution to an appropriate anti-solvent at a given temperature induced solid formation. The resulting solids were isolated by vacuum filtration and air-dried prior to analysis.

Slurry Experiments: Enough Compound I was added to a given solvent so that undissolved solids were present. The mixture was then agitated in a sealed vial at ambient temperature using either an orbital shaker or a rotating wheel. After several days the solids were isolated by vacuum filtration and allowed to dry at ambient temperature in the fume hood with the cap for the sample vial loosened.

Relative Humidity (RH) Stress: Open vials containing solid samples were placed inside chambers containing saturated salt solutions along with a small amount of the undissolved salt. The chambers were sealed and allowed to stand at ambient temperature for several weeks. Samples were analyzed by X-ray powder diffraction (XRPD) immediately after removing the sample from the RH chamber. The RH values for these salt solutions were obtained from an ASTM standard. See a) “Preparation and Use of Relative Humidity Chambers” SSCI Method MTD 032.01 Mar. 3, 2003; and b) “Standard Practice for Maintaining Constant Relative Humidity by Means of Aqueous Solutions” ASTM Standard E 104-02 (2002).

Vapor Stress: Open vials containing solid samples were placed inside chambers containing a specified solvent. The chambers were sealed and allowed to stand at ambient temperature for several weeks. Samples were analyzed by X-ray powder diffraction (XRPD) immediately after removing the sample from the vapor chamber.

Elevated Temperature Slurry Experiments: Solutions of Compound I were prepared by adding enough solids to a given solvent so that undissolved solids were present. The mixture was then agitated in a sealed vial at elevated temperature using an orbital shaker. After several days the solids were isolated by vacuum filtration and allowed to dry at ambient temperature in the fume hood with the cap of the sample vial loosened.

Milling Experiments: A sample of compound I was ground at room temperature using a ball mill (Retsch Mixer Mill model MM200) for a total of 20 minutes at a frequency of 30 Hz.

Lyophilization (Freeze Drying): A solution of Compound I in water was prepared in a round bottom flask, the solution was frozen in a dry ice/acetone bath and placed on a commercial freeze dryer equipped with a rotary vane mechanical vacuum pump. The flask was wrapped in glass wool and aluminum foil to insulate the frozen solution during the freeze drying operation.

Melt/Cool Under Vacuum (Sample No. 1888-61-01): A sufficient amount of material was placed in a round bottomed flask and placed under vacuum. The flask was heated to 168° C. in a preheated oil bath. After the melt of the solids (which took several minutes), the flask was removed from the oil bath and cooled to room temperature while still under vacuum. The solids were collected, lightly ground and stored in a vial.

### Example 3

### Slurry Interconversion Studies

In order to determine the relative stability of the polymorphs Compound I form A was slurried in separate experiments with roughly equal amounts of forms B, C and E in methanol at room temperature. After 12 days the solids were isolated and form A was recovered as the exclusive solid phase (Table 6). In addition to these experiments, slurries of forms B, C and D in the absence of other solid phases provided form A when slurried for 5 days in methanol at room temperature. These experiments demonstrate that form A is more stable than form B, C, D, or E in methanol at room temperature. It should be noted that the hydration states of these solid phases may be different for the different forms, which precludes an analysis of the true thermodynamic stability ordering through slurry experiments.

TABLE 6
Slurry Inter-conversion Experiments in Methanol at Room Temperature

| Starting Solid | Slurry Time | XRPD |
|---|---|---|
| Phase(s) | (days) | Result |
| Form B | 5 | Form A |
| Form C | 5 | Form A |
| Form D | 5 | Form A |
| Forms A and B | 12 | Form A |
| Forms A and C | 12 | Form A |
| Forms A and E | 12 | Form A |

### Example 4

### X-Ray Powder Diffraction

X-ray powder diffraction analyses were performed using an Inel XRG-3000 diffractometer equipped with a CPS (Curved Position Sensitive) detector with a 2 Orange of 120°. The instrument calibration was performed using a silicon reference standard. The tube voltage and amperage were set to 40 kV and 30 mA, respectively. The monochromator slit was set at 5 mm by 160 μm. Samples were placed in an aluminum sample holder with a silicon insert or in glass XRPD-quality capillaries. Each capillary was mounted onto a goniometer head that is motorized to permit spinning of the capillary during data acquisition. Real time data were collected using Cu—Kα radiation at a resolution of 0.03° 2θ. Typically, the data were collected over a period of 300 seconds. Only the data points within the range of 2.5-40° 2θ are displayed in the plotted XRPD patterns.

### Example 5

### Thermogravimetric Analysis (TGA)

Thermogravimetric analyses (TGA) were carried out on a TA Instruments 2950 thermogravimetric analyzer. The calibration standards were nickel and Alumel™. Samples were placed in an aluminum sample pan, inserted into the TG furnace, and accurately weighed. The samples were heated in a stream of nitrogen at a rate of 10° C./min. The temperature of the furnace was equilibrated at 25° C. prior to the analysis of the samples. The percent mass loss values provided in the tables are rounded to the nearest 0.1%.

### Example 6

### Differential Scanning Calorimetry (DSC)

Differential scanning calorimetry (DSC) analyses were carried out on a TA Instruments differential scanning calorimeter 2920. Accurately weighed samples were placed in either crimped pans, crimped pans with manual pinhole or uncrimped pans to allow for pressure release. Each sample was heated under nitrogen at a rate of 10° C./min to 350° C. Indium metal was used as the calibration standard. Temperatures are reported at the transition maxima and reported to one decimal place, and the integrated events are expressed in J/g and rounded to the nearest whole number.

Cyclic DSC experiments were carried out by placing accurately weighed samples in uncrimped pans. Samples were heated under nitrogen at a rate of 10° C./min to either 150 or 180° C. subsequently cooled to −40° C. This procedure was repeated twice before the sample was heated to 250° C.

### Example 7

### Hot-Stage Microscopy

Hot-stage microscopy was carried out using a Wagner & Munz apparatus consisting of a Kofler stage mounted on a Leica Microscope. The stage temperature was calibrated using vanillin and caffeine USP standards each day prior to running samples. For each sample, a small quantity was placed on a microscope slide and covered. Samples were heated at approximately 4° C./min and images were captured periodically using a 10× objective lens and a CCD camera. A cross-polarizing filter was used to observe birefringence.

### Example 8

### Fourier Transform Infrared Spectroscopy (FT-IR)

Infrared spectra were acquired on a Magna-IR 860® Fourier transform infrared (FT-IR) spectrophotometer (Thermo Nicolet) equipped with an Ever-Glo mid/far IR source, an extended range potassium bromide (KBr) beamsplitter, and a deuterated triglycine sulfate (DTGS) detector. An ATR Thunderdome accessory with non-concave tip was used for sampling. Each spectrum represents 256 co-added scans collected from 4000-675 cm⁻¹ at a spectral resolution of 4 cm⁻¹. A clean Ge crystal background data set was acquired. A Log 1/R (R=reflectance) spectrum was acquired by taking a ratio of these two data sets against each other. Wavelength calibration was performed using polystyrene.

### Example 9

### Raman Spectroscopy

FT-Raman spectra were acquired on a Raman accessory module interfaced to a Magna 860® Fourier transform infrared (FT-IR) spectrophotometer (Thermo Nicolet). This module uses an excitation wavelength of 1064 nm and an indium gallium arsenide (InGaAs) detector. A Nd:YVO₄ laser was used to irradiate the samples using approximate laser powers in the range of 0.4 to 0.8 W. The samples were prepared for analysis by placing the material in a glass capillary tube. A total of 256 sample scans were collected at a spectral resolution of 4 cm⁻¹ using Happ-Genzel apodization. Wavelength calibration was performed using sulfur and cyclohexane.

### Example 10

### NMR Spectroscopy

Solution ¹H NMR spectra were acquired at ambient temperature on a Bruker Instruments AM-250 spectrometer at a magnetic field strength of 5.87 Tesla (¹H Larmor frequency=250 MHz). The samples were dissolved in NMR-grade DMSO-d₆. Spectra were acquired with a ¹H pulse width of 8.5 μs (90°), a 2.5 second acquisition time, a 5.0 second delay between scans, a spectral width of 6400.0 Hz with 32K data points, and 32 co-added scans. Each free induction decay (FID) was processed with GRAMS/32 AI software v. 6.00 using a Fourier number equal to twice the number of acquired points [or a larger multiple if zero filling was used] with an exponential line broadening factor of 0.2 Hz to improve sensitivity. Peak tables were generated by the GRAMS software peak picking algorithm. For these spectra the residual peak from incompletely deuterated DMSO-d₆ is located at approximately 2.50 ppm.

### Example 11

### Moisture Sorption/Desorption Analyses

Moisture sorption/desorption data were collected on a VTI SGA-100 Vapor Sorption Analyzer. Sorption and desorption data were collected over a range of 5% to 95% relative humidity (RH) at 10% RH intervals under a nitrogen purge. Sodium chloride (NaCl) and polyvinylpyrrolidone (PVP) were used as the calibration standards. Equilibrium criteria used for analysis were less than 0.0100% weight change in 5 minutes, with a maximum equilibration time of 180 minutes if the weight criterion was not met. The plotted data that contain the percent weight change values at various RH intervals are relative to the initial weight of the sample. The plotted data have not been corrected for the initial moisture content. However, the weight gain data provided in the tables for the various relative humidity ranges are calculated using the sample weight at the beginning of the particular RH event as the initial weight. In particular, the equilibration event wt. % change from initial wt. to 5% RH was calculated as [(wt at 5% RH−initial wt.)/initial wt.]*100; and the sorption event wt. % change from 5% RH to 95% RH was calculated as [(wt. at 95% RH−wt at 5% RH)/wt at 5% RH]*100; and the desorption event wt. % change from 95% RH to 5% RH was calculated as [(wt. at 5% RH−wt at 95% RH)/wt at 95% RH]*100. This type of analysis permits direct comparison of the experimental weight changes to the theoretical values predicted for the inter-conversion of anhydrates to hydrated forms.

### Example 12

### Karl Fischer Water Analysis

Karl Fischer (titrimetric) water analysis was performed by Galbraith Laboratories, Inc. of Knoxville, Tenn. according to U.S. Pharmacopoeia, vol. 24, method 921, U.S.P. Pharmacopeial Convention, Inc, Rockville, Md.. The polymorph was tested for water content by Karl Fischer titration using a coulometer according to the published procedure and the manufacturer's coulometer instructions.

While the present invention is disclosed with reference to certain embodiments and examples detailed above, it is to be understood that these embodiments and examples are intended to be illustrative rather than limiting. As such, it is contemplated that various modifications and variations will be apparent to those skilled in the art and intended that those modifications and variations fall within the scope of the invention and the appended claims. All patents, patent applications, papers, and books cited in this application are incorporated by reference herein in their entirety.

What is claimed is:

1. A composition comprising Compound I wherein at least a portion of Compound I is present in polymorphic Form A characterized by one or more physical properties selected from the group consisting of:
an X-ray powder diffraction pattern with salient features being major diffraction lines at follows:

| | | | | | | | | |
|---|---|---|---|---|---|---|---|---|
| °2θ | 6.22 | 7.92 | 8.51 | 12.77 | 14.72 | 15.48 | 29.23 | 32.44 |

using Cu—Kα radiation;
an IR spectrum comprising absorption peaks at 3478, 3383, 3053, 2928, 2843, 1671, 1105 and 997 cm⁻¹;
a Raman spectrum comprising peaks at 3099, 3077, 1458, 1333, 889, 702, 651 and 615 cm⁻¹; and/or
a differential scanning calorimetry spectrum having an endotherm range of about 160° C. to about 185° C.

2. The composition according to claim 1, wherein greater than 50% of Compound I (by weight) is present in the composition as Form A.

3. A pharmaceutical composition comprising:
Compound I wherein at least a portion of Compound I is present in polymorphic Form A characterized by one or more physical properties selected from the group consisting of an X-ray powder diffraction pattern with salient features being major diffraction lines at follows:

| | | | | | | | | |
|---|---|---|---|---|---|---|---|---|
| °2θ | 6.22 | 7.92 | 8.51 | 12.77 | 14.72 | 15.48 | 29.23 | 32.44 |

using Cu Kα radiation;
an IR spectrum comprising absorption peaks at 3478, 3383, 3053, 2928, 2843, 1671, 1105 and 997 cm⁻¹;
a Raman spectrum comprising peaks at 3099, 3077, 1458, 1333, 889, 702, 651 and 615 cm⁻¹; and/or
a differential scanning calorimetry spectrum having an endotherm range of about 160° C. to about 185° C.; and
one or more pharmaceutical carriers.

4. The pharmaceutical composition according to claim 3, wherein greater than 50% of Compound I (by weight) is present in the composition as Form A.

5. The pharmaceutical composition according to claim 3, wherein the composition is in an oral dosage form selected from the group consisting of pills, tablets, capsules, emulsions, suspensions, microsuspensions, wafers, sprinkles, chewing gum, powders, lyophilized powders, granules, and troches.

6. The pharmaceutical composition according to claim 3, wherein the composition is in a parenteral dosage form selected from the group consisting of suspensions, microsuspensions, emulsions, solid forms suitable for suspension or emulsification prior to injection, and implantable devices.

7. The pharmaceutical composition according to claim 3, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

8. The pharmaceutical composition according to claim 3, wherein the composition is in a pulmonary dosage form selected from the group consisting of powders, aerosols, suspensions, microsuspensions, and emulsions.

9. A kit comprising:
a pharmaceutical composition comprising Compound I wherein at least a portion of Compound I is present in polymorphic Form A characterized by one or more physical properties selected from the group consisting of
an X-ray powder diffraction pattern with salient features being major diffraction lines at follows:

| | | | | | | | | |
|---|---|---|---|---|---|---|---|---|
| °2θ | 6.22 | 7.92 | 8.51 | 12.77 | 14.72 | 15.48 | 29.23 | 32.44 |

using Cu—Kα radiation;
an IR spectrum comprising absorption peaks at 3478, 3383, 3053, 2928, 2843, 1671, 1105 and 997 cm⁻¹;
a Raman spectrum comprising peaks at 3099, 3077, 1458, 1333, 889, 702, 651 and 615 cm⁻¹; and/or
a differential scanning calorimetry spectrum having an endotherm range of about 160° C. to about 185° C.; and
instructions which comprise one or more forms of information selected from the group consisting of indicating a disease state for which the composition is to be administered, storage information for the composition, dosing information and instructions regarding how to administer the composition.

10. An article of manufacture comprising:
a composition according to claim 1; and
packaging materials.

11. A therapeutic method comprising administering a composition according to claim 1 to a subject.

12. A method of inhibiting dipeptidyl peptidases comprising causing a composition according to claim 1 to be present in a subject.

13. A method of treating a disease state for which dipeptidyl peptidases possess activity that contributes to the pathology and/or symptomology of the disease state, the method comprising administering a composition according to claim 1 to a subject.

14. A method for preparing Compound I wherein at least a portion of Compound I is present in polymorph Form A, the method comprising: crystallization from any of the following solvent systems comprising (i) acetone and water; (ii) methanol; (iii) methanol and acetone; (iv) methanol and toluene; and (v) water.

15. A composition comprising Compound I wherein at least a portion of Compound I is present in polymorphic Form B characterized by one or more physical properties selected from the group consisting of
an X-ray powder diffraction pattern with salient features being major diffraction lines as follows:

| | | | | | |
|---|---|---|---|---|---|
| °2θ | 3.11 | 10.96 | 14.07 | 20.04 | 20.82 |

an IR spectrum comprising absorption peaks at 2951, 1506, 1420, 1217, 1161, 1115 and 1033 cm⁻¹;
a Raman spectrum comprising peaks at 3084, 2993, 2926, 1686 and 1540 cm⁻¹; and/or
a differential scanning calorimetry spectrum having an endotherm range of about 100° C. to about 135° C.

16. The composition according to claim 15, wherein greater than 50% of Compound I (by weight) is present in the composition as Form B.

17. A pharmaceutical composition comprising:
Compound I wherein at least a portion of Compound I is present in polymorphic Form B characterized by one or more physical properties selected from the group consisting of an X-ray powder diffraction pattern with salient features being major diffraction lines as follows:

| | | | | | |
|---|---|---|---|---|---|
| °2θ | 3.11 | 10.96 | 14.07 | 20.04 | 20.82 |

an IR spectrum comprising absorption peaks at 2951, 1506, 1420, 1217, 1161, 1115 and 1033 cm⁻¹;
a Raman spectrum comprising peaks at 3084, 2993, 2926, 1686 and 1540 cm⁻¹; and/or a differential scanning calorimetry spectrum having an endotherm range of about 100° C. to about 135° C.; and
one or more pharmaceutical carriers.

18. The pharmaceutical composition according to claim 17, wherein greater than 50% of Compound I (by weight) is present in the composition as Form B.

19. The pharmaceutical composition according to claim 17, wherein the composition is in an oral dosage form selected from the group consisting of pills, tablets, capsules, emulsions, suspensions, microsuspensions, wafers, sprinkles, chewing gum, powders, lyophilized powders, granules, and troches.

20. The pharmaceutical composition according to claim 17, wherein the composition is in a parenteral dosage form selected from the group consisting of suspensions, microsuspensions, emulsions, solid forms suitable for suspension or emulsification prior to injection, and implantable devices.

21. The pharmaceutical composition according to claim 17, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

22. The pharmaceutical composition according to claim 17, wherein the composition is in a pulmonary dosage form selected from the group consisting of powders, aerosols, suspensions, microsuspensions, and emulsions.

23. A kit comprising:
a pharmaceutical composition comprising Compound I wherein at least a portion of Compound I is present in polymorphic Form B characterized by one or more physical properties selected from the group consisting of
an X-ray powder diffraction pattern with salient features being major diffraction lines as follows:

| | | | | | |
|---|---|---|---|---|---|
| °2θ | 3.11 | 10.96 | 14.07 | 20.04 | 20.82 |

an IR spectrum comprising absorption peaks at 2951, 1506, 1420, 1217, 1161, 1115 and 1033 cm⁻¹;
a Raman spectrum comprising peaks at 3084, 2993, 2926, 1686 and 1540 cm⁻¹; and/or
a differential scanning calorimetry spectrum having an endotherm range of about 100° C. to about 135° C.; and
instructions which comprise one or more forms of information selected from the group consisting of indicating a disease state for which the composition is to be administered, storage information for the composition, dosing information and instructions regarding how to administer the composition.

24. An article of manufacture comprising:
a composition according to claim 15; and
packaging materials.

25. A therapeutic method comprising administering a composition according to claim 15 to a subject.

26. A method of inhibiting dipeptidyl peptidases comprising causing a composition according to claim 15 to be present in a subject.

27. A method of treating a disease state for which dipeptidyl peptidases possess activity that contributes to the pathology and/or symptomology of the disease state, the method comprising administering a composition according to claim 15 to a subject.

28. A method for preparing Compound I wherein at least a portion of Compound I is present in polymorph Form B, the method comprising: crystallization from any of the following solvent systems comprising (i) tetrahydrofuran, (ii) dioxane and water; and (iii) acetonitrile and water.

29. A composition comprising Compound I wherein at least a portion of Compound I is present in polymorphic Form C characterized by one or more physical properties selected from the group consisting of
an X-ray powder diffraction pattern with salient features being major diffraction lines as follows:

| | | | | | |
|---|---|---|---|---|---|
| °2θ | 5.22 | 14.29 | 15.68 | 18.04 | 29.58 |

an IR spectrum comprising absorption peaks at 3513, 2958, 1723, 1635, 1587, 813 and 777 cm⁻¹;
a Raman spectrum comprising peaks at 1944, 1436, 1419, 1372, 1297, 979 and 487 cm⁻¹; and/or
a differential scanning calorimetry spectrum having an endotherm range of about 100° C. to about 135° C.

30. The composition according to claim 29, wherein greater than 50% of Compound I (by weight) is present in the composition as Form C.

31. A pharmaceutical composition comprising:
Compound I wherein at least a portion of Compound I is present in polymorphic Form C characterized by one or more physical properties selected from the group consisting of an X-ray powder diffraction pattern with salient features being major diffraction lines as follows:

| | | | | | |
|---|---|---|---|---|---|
| °2θ | 5.22 | 14.29 | 15.68 | 18.04 | 29.58 |

an IR spectrum comprising absorption peaks at 3513, 2958, 1723, 1635, 1587, 813 and 777 cm⁻¹;
a Raman spectrum comprising peaks at 1944, 1436, 1419, 1372, 1297, 979 and 487 cm⁻¹; and/or
a differential scanning calorimetry spectrum having an endotherm range of about 100° C. to about 135° C.; and
one or more pharmaceutical carriers.

32. The pharmaceutical composition according to claim 31, wherein greater than 50% of Compound I (by weight) is present in the composition as Form C.

33. The pharmaceutical composition according to claim 31, wherein the composition is in an oral dosage form selected from the group consisting of pills, tablets, capsules, emulsions, suspensions, microsuspensions, wafers, sprinkles, chewing gum, powders, lyophilized powders, granules, and troches.

34. The pharmaceutical composition according to claim 31, wherein the composition is in a parenteral dosage form selected from the group consisting of suspensions, microsuspensions, emulsions, solid forms suitable for suspension or emulsification prior to injection, and implantable devices.

35. The pharmaceutical composition according to claim 31, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

36. The pharmaceutical composition according to claim 31, wherein the composition is in a pulmonary dosage form selected from the group consisting of powders, aerosols, suspensions, microsuspensions, and emulsions.

37. A kit comprising:
a pharmaceutical composition comprising Compound I wherein at least a portion of Compound I is present in polymorphic Form C characterized by one or more physical properties selected from the group consisting of:
an X-ray powder diffraction pattern with salient features being major diffraction lines at follows:

| | | | | | |
|---|---|---|---|---|---|
| °2θ | 5.22 | 14.29 | 15.68 | 18.04 | 29.58 |

an IR spectrum comprising absorption peaks at 3513, 2958, 1723, 1635, 1587, 813 and 777 cm⁻¹;
a Raman spectrum comprising peaks at 1944, 1436, 1419, 1372, 1297, 979 and 487 cm⁻¹; and/or
has a differential scanning calorimetry spectrum having an endotherm range of about 100° C. to about 135° C.; and
instructions which comprise one or more forms of information selected from the group consisting of indicating a disease state for which the composition is to be administered, storage information for the composition, dosing information and instructions regarding how to administer the composition.

38. An article of manufacture comprising:
a composition according to claim 29; and
packaging materials.

39. A therapeutic method comprising administering a composition according to claim 29 to a subject.

40. A method of inhibiting dipeptidyl peptidases comprising causing a composition according to claim 29 to be present in a subject.

41. A method of treating a disease state for which dipeptidyl peptidases possess activity that contributes to the pathology and/or symptomology of the disease state, the method comprising administering a composition according to claim 29 to a subject.

42. A method for preparing Compound I wherein at least a portion of Compound I is present in polymorph Form C, the method comprising: crystallization from any of the following solvent systems comprising (i) ethanol and water, and (ii) isopropanol and water.

43. A composition comprising Compound I wherein at least a portion of Compound I is present in polymorphic Form D characterized by an X-ray powder diffraction pattern with salient features being major diffraction lines as follows:

| | | | | |
|---|---|---|---|---|
| °2θ | 6.21 | 7.90 | 8.50 | 10.58 |

44. The composition according to claim 43, wherein greater than 50% of Compound I (by weight) is present in the composition as Form D.

45. A pharmaceutical composition comprising:
Compound I wherein at least a portion of Compound I is present in polymorphic Form D characterized by an X-ray powder diffraction pattern with salient features being major diffraction lines as follows:

| | | | | |
|---|---|---|---|---|
| °2θ | 6.21 | 7.90 | 8.50 | 10.58 |

and one or more pharmaceutical carriers.

46. The pharmaceutical composition according to claim 45, wherein greater than 50% of Compound I (by weight) is present in the composition as Form D.

47. The pharmaceutical composition according to claim 45, wherein the composition is in an oral dosage form selected from the group consisting of pills, tablets, capsules, emulsions, suspensions, microsuspensions, wafers, sprinkles, chewing gum, powders, lyophilized powders, granules, and troches.

48. The pharmaceutical composition according to claim 45, wherein the composition is in a parenteral dosage form selected from the group consisting of suspensions, microsuspensions, emulsions, solid forms suitable for suspension or emulsification prior to injection, and implantable devices.

49. The pharmaceutical composition according to claim 45, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

50. The pharmaceutical composition according to claim 45, wherein the composition is in a pulmonary dosage form selected from the group consisting of powders, aerosols, suspensions, microsuspensions, and emulsions.

51. A kit comprising:
a pharmaceutical composition comprising Compound I wherein at least a portion of Compound I is present in polymorphic Form D characterized by an X-ray powder diffraction pattern with salient features being major diffraction lines as follows:

| | | | | |
|---|---|---|---|---|
| °2θ | 6.21 | 7.90 | 8.50 | 10.58 |

and instructions which comprise one or more forms of information selected from the group consisting of indicating a disease state for which the composition is to be administered, storage information for the composition, dosing information and instructions regarding how to administer the composition.

52. An article of manufacture comprising:
a composition according to claim 43; and
packaging materials.

53. A therapeutic method comprising administering a composition according to claim 43 to a subject.

54. A method of inhibiting dipeptidyl peptidases comprising causing a composition according to claim 43 to be present in a subject.

55. A method of treating a disease state for which dipeptidyl peptidases possess activity that contributes to the pathology and/or symptomology of the disease state, the method comprising administering a composition according to claim 43 to a subject.

56. A method for preparing Compound I wherein at least a portion of Compound I is present in polymorph Form D, the method comprising: crystallization from any of the following solvent systems comprising (i) water, and (ii) methanol.

57. A composition comprising Compound I wherein at least a portion of Compound I is present in polymorphic Form E characterized by one or more physical properties selected from the group consisting of
an X-ray powder diffraction pattern with salient features being major diffraction lines as follows:

| | | | | | | | | | | |
|---|---|---|---|---|---|---|---|---|---|---|
| °2θ | 5.51 | 9.66 | 11.03 | 13.83 | 14.41 | 16.50 | 18.83 | 22.51 | 28.15 | 29.47 |

an IR spectrum comprising absorption peaks at 3440, 3330, 3108, 1580 and 1381 cm⁻¹;
a Raman spectrum comprising peaks at 3069, 1286 and 1236 cm⁻¹; and/or a differential scanning calorimetry spectrum having an endotherm range of about 100° C. to about 133° C.

58. The composition according to claim 57, wherein greater than 50% of Compound I (by weight) is present in the composition as Form E.

59. A pharmaceutical composition comprising:
Compound I wherein at least a portion of Compound I is present in polymorphic Form E characterized by one or more physical properties selected from the group consisting of
an X-ray powder diffraction pattern with salient features being major diffraction lines as follows:

| | | | | | | | | | | |
|---|---|---|---|---|---|---|---|---|---|---|
| °2θ | 5.51 | 9.66 | 11.03 | 13.83 | 14.41 | 16.50 | 18.83 | 22.51 | 28.15 | 29.47 |

an IR spectrum comprising absorption peaks at 3440, 3330, 3108, 1580 and 1381 cm⁻¹;
a Raman spectrum comprising peaks at 3069, 1286 and 1236 cm⁻¹; and/or
a differential scanning calorimetry spectrum having an endotherm range of about 100° C. to about 133° C.; and
one or more pharmaceutical carriers.

60. The pharmaceutical composition according to claim 59, wherein greater than 50% of Compound I (by weight) is present in the composition as Form E.

61. The pharmaceutical composition according to claim 59, wherein the composition is in an oral dosage form selected from the group consisting of pills, tablets, capsules, emulsions, suspensions, microsuspensions, wafers, sprinkles, chewing gum, powders, lyophilized powders, granules, and troches.

62. The pharmaceutical composition according to claim 59, wherein the composition is in a parenteral dosage form selected from the group consisting of suspensions, microsuspensions, emulsions, solid forms suitable for suspension or emulsification prior to injection, and implantable devices.

63. The pharmaceutical composition according to claim 59, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

64. The pharmaceutical composition according to claim 59, wherein the composition is in a pulmonary dosage form selected from the group consisting of powders, aerosols, suspensions, microsuspensions, and emulsions.

65. A kit comprising:
a pharmaceutical composition comprising Compound I wherein at least a portion of Compound I is present in polymorphic Form E characterized by one or more physical properties selected from the group consisting of:
an X-ray powder diffraction pattern with salient features being major diffraction lines at follows:

| | | | | | | | | | | |
|---|---|---|---|---|---|---|---|---|---|---|
| °2θ | 5.51 | 9.66 | 11.03 | 13.83 | 14.41 | 16.50 | 18.83 | 22.51 | 28.15 | 29.47 |

an IR spectrum comprising absorption peaks at 3440, 3330, 3108, 1580 and 1381 cm⁻¹;
a Raman spectrum comprising peaks at 3069, 1286 and 1236 cm⁻¹; and/or a differential scanning calorimetry spectrum having an endotherm range of about 100° C. to about 133° C.; and
instructions which comprise one or more forms of information selected from the group consisting of indicating a disease state for which the composition is to be administered, storage information for the composition, dosing information and instructions regarding how to administer the composition.

66. An article of manufacture comprising:
a composition according to claim 57; and
packaging materials.

67. A therapeutic method comprising administering a composition according to claim 57 to a subject.

68. A method of inhibiting dipeptidyl peptidases comprising causing a composition according to claim 57 to be present in a subject.

69. A method of treating a disease state for which dipeptidyl peptidases possess activity that contributes to the pathology and/or symptomology of the disease state, the method comprising administering a composition according to claim 57 to a subject.

70. A method for preparing Compound I wherein at least a portion of Compound I is present in polymorph Form E, the method comprising: crystallization from any of the following solvent systems comprising (i) water and acetonitrile, and (ii) water and dioxane.

71. A composition comprising Compound I wherein at least a portion of Compound I is present in polymorphic Form F characterized by an X-ray powder diffraction pattern with salient features being major diffraction lines as follows:

| | | | | |
|---|---|---|---|---|
| °2θ | 10.79 | 11.59 | 12.51 | 13.02 |

72. The composition according to claim 71, wherein greater than 50% of Compound I (by weight) is present in the composition as Form F.

73. A pharmaceutical composition comprising:
Compound I wherein at least a portion of Compound I is present in polymorphic Form F characterized by an X-ray powder diffraction pattern with salient features being major diffraction lines as follows:

| | | | | |
|---|---|---|---|---|
| °2θ | 10.79 | 11.59 | 12.51 | 13.02 |

and one or more pharmaceutical carriers.

74. The pharmaceutical composition according to claim 73, wherein greater than 50% of Compound I (by weight) is present in the composition as Form F.

75. The pharmaceutical composition according to claim 73, wherein the composition is in an oral dosage form selected from the group consisting of pills, tablets, capsules, emulsions, suspensions, microsuspensions, wafers, sprinkles, chewing gum, powders, lyophilized powders, granules, and troches.

76. The pharmaceutical composition according to claim 73, wherein the composition is in a parenteral dosage form selected from the group consisting of suspensions, microsuspensions, emulsions, solid forms suitable for suspension or emulsification prior to injection, and implantable devices.

77. The pharmaceutical composition according to claim 73, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

78. The pharmaceutical composition according to claim 73, wherein the composition is in a pulmonary dosage form selected from the group consisting of powders, aerosols, suspensions, microsuspensions, and emulsions.

79. A kit comprising:
a pharmaceutical composition comprising Compound I wherein at least a portion of Compound I is present in polymorphic Form F characterized by an X-ray powder diffraction pattern with salient features being major diffraction lines as follows:

| | | | | |
|---|---|---|---|---|
| °2θ | 10.79 | 11.59 | 12.51 | 13.02 |

and instructions which comprise one or more forms of information selected from the group consisting of indicating a disease state for which the composition is to be administered, storage information for the composition, dosing information and instructions regarding how to administer the composition.

80. An article of manufacture comprising:
a composition according to claim 71; and
packaging materials.

81. A therapeutic method comprising administering a composition according to claim 71 to a subject.

82. A method of inhibiting dipeptidyl peptidases comprising causing a composition according to claim 71 to be present in a subject.

83. A method of treating a disease state for which dipeptidyl peptidases possess activity that contributes to the pathology and/or symptomology of the disease state, the method comprising administering a composition according to claim 71 to a subject.

84. A method for preparing Compound I wherein at least a portion of Compound I is present in polymorph Form F, the method comprising crystallization from water.

85. A composition comprising Compound I wherein at least a portion of Compound I is present in amorphic Form 1, characterized by one or more physical properties selected from the group consisting of:
an IR spectrum comprising absorption peaks at 3407, 1536 and 1287 cm⁻¹;
a Raman spectrum comprising absorption peaks at 3074, 1789 and 1398 cm⁻¹; and
a differential scanning calorimetry spectrum having an endotherm range of about 175° C. to about 238° C.

86. The composition according to claim 85, wherein greater than 50% of Compound I (by weight) is present in the composition as amorphic Form 1.

87. A pharmaceutical composition comprising:
Compound I wherein at least a portion of Compound I is present in amorphic Form 1 characterized by one or more physical properties selected from the group consisting of:
an IR spectrum comprising absorption peaks at 3407, 1536 and 1287 cm⁻¹;
a Raman spectrum comprising absorption peaks at 3074, 1789 and 1398 cm⁻¹; and
a differential scanning calorimetry spectrum having an endotherm range of about 175° C. to about 238° C.; and
one or more pharmaceutical carriers.

88. The pharmaceutical composition according to claim 87, wherein greater than 50% of Compound I (by weight) is present in the composition as amorphic Form 1.

89. The pharmaceutical composition according to claim 87, wherein the composition is in an oral dosage form selected from the group consisting of pills, tablets, capsules, emulsions, suspensions, microsuspensions, wafers, sprinkles, chewing gum, powders, lyophilized powders, granules, and troches.

90. The pharmaceutical composition according to claim 87, wherein the composition is in a parenteral dosage form selected from the group consisting of suspensions, microsuspensions, emulsions, solid forms suitable for suspension or emulsification prior to injection, and implantable devices.

91. The pharmaceutical composition according to claim 87, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

92. The pharmaceutical composition according to claim 87, wherein the composition is in a pulmonary dosage form selected from the group consisting of powders, aerosols, suspensions, microsuspensions, and emulsions.

93. A kit comprising:
a pharmaceutical composition comprising Compound I wherein at least a portion of Compound I is present in amorphic Form 1 characterized by one or more physical properties selected from the group consisting of:
an IR spectrum comprising absorption peaks at 3407, 1536 and 1287 cm⁻¹;
a Raman spectrum comprising absorption peaks at 3074, 1789 and 1398 cm⁻¹; and
a differential scanning calorimetry spectrum having an endotherm range of about 175° C. to about 238° C.; and
instructions which comprise one or more forms of information selected from the group consisting of indicating a disease state for which the composition is to be administered, storage information for the composition, dosing information and instructions regarding how to administer the composition.

94. An article of manufacture comprising:
a composition according to claim 85; and
packaging materials.

95. A therapeutic method comprising administering a composition according to claim 85 to a subject.

96. A method of inhibiting dipeptidyl peptidases comprising causing a composition according to claim 85 to be present in a subject.

97. A method of treating a disease state for which dipeptidyl peptidases possess activity that contributes to the pathology and/or symptomology of the disease state, the method comprising administering a composition according to claim 85 to a subject.

98. A method for preparing Compound I wherein at least a portion of Compound I is present in amorphic Form 1, the method comprising freeze drying Compound I from water.

99. A composition comprising Compound I wherein at least a portion of Compound I is present in amorphic Form 2, characterized by one or more physical properties selected from the group consisting of:
an IR spectrum comprising absorption peaks at 3407, 1536 and 1287 cm⁻¹;
a Raman spectrum comprising absorption peaks at 3074, 1789 and 1398 cm⁻¹; and
has a differential scanning calorimetry spectrum having a first endotherm range of about 50° C. to about 88° C., and a second endotherm range of about 182° C. to about 230° C.

100. The composition according to claim 99, wherein greater than 50% of Compound I (by weight) is present in the composition as amorphic Form 2.

101. A pharmaceutical composition comprising:
Compound I wherein at least a portion of Compound I is present in polymorphic amorphic Form 2 characterized by one or more physical properties selected from the group consisting of:
an IR spectrum comprising absorption peaks at 3407, 1536 and 1287 cm⁻¹;
a Raman spectrum comprising absorption peaks at 3074, 1789 and 1398 cm⁻¹; and
has a differential scanning calorimetry spectrum having a first endotherm range of about 50° C. to about 88° C., and a second endotherm range of about 182° C. to about 230° C.; and
one or more pharmaceutical carriers.

102. The pharmaceutical composition according to claim 101, wherein greater than 50% of Compound I (by weight) is present in the composition as amorphic Form 2.

103. The pharmaceutical composition according to claim 101, wherein the composition is in an oral dosage form selected from the group consisting of pills, tablets, capsules, emulsions, suspensions, microsuspensions, wafers, sprinkles, chewing gum, powders, lyophilized powders, granules, and troches.

104. The pharmaceutical composition according to claim 101, wherein the composition is in a parenteral dosage form selected from the group consisting of suspensions, microsuspensions, emulsions, solid forms suitable for suspension or emulsification prior to injection, and implantable devices.

105. The pharmaceutical composition according to claim 101, wherein the composition is in a topical or transdermal dosage form selected from the group consisting of suspensions, microsuspensions, emulsions, creams, gels, ointments, lotions, tinctures, pastes, powders, foams, aerosols, irrigations, sprays, suppositories, bandages, and dermal patches.

106. The pharmaceutical composition according to claim 101, wherein the composition is in a pulmonary dosage form selected from the group consisting of powders, aerosols, suspensions, microsuspensions, and emulsions.

107. A kit comprising:
a pharmaceutical composition comprising Compound I wherein at least a portion of Compound I is present in amorphic Form 2 characterized by one or more physical properties selected from the group consisting of:
an IR spectrum comprising absorption peaks at 3407, 1536 and 1287 cm⁻¹;
a Raman spectrum comprising absorption peaks at 3074, 1789 and 1398 cm⁻¹; and
has a differential scanning calorimetry spectrum having a first endotherm range of about 50° C. to about 88° C., and a second endotherm range of about 182° C. to about 230° C.; and
instructions which comprise one or more forms of information selected from the group consisting of indicating a disease state for which the composition is to be administered, storage information for the composition, dosing information and instructions regarding how to administer the composition.

108. An article of manufacture comprising:
a composition according to claim 99; and
packaging materials.

109. A therapeutic method comprising administering a composition according to claim 99 to a subject.

110. A method of inhibiting dipeptidyl peptidases comprising causing a composition according to claim 99 to be present in a subject.

111. A method of treating a disease state for which dipeptidyl peptidases possess activity that contributes to the pathology and/or symptomology of the disease state, the method comprising administering a composition according to claim 99 to a subject.

112. A method for preparing Compound I wherein at least a portion of Compound I is present in amorphic Form 2, the method comprising milling of Compound I.
